(19) United States
(12) Patent Application Publication
(10) Pub. No.: US 2010/0076787 A1
(43) Pub. Date: Mar. 25, 2010

(54) METHOD FOR PREPARING A MEDICAL DATA REPORT

(76) Inventors: Stephen Naylor, Rochester, MN (US); Brian Kleber, Columbus, IN (US); Christa Curtis, Bloomington, IN (US); Autumn Workman, Bloomington, IN (US); Scott Beason, Bloomington, IN (US)

Correspondence Address: TAFT, STETTINIUS & HOLLISTER LLP, SUITE 1800, 425 WALNUT STREET, CINCINNATI, OH, 45202-3957, US

(21) Appl. No.: 12/558,037

(22) Filed: Sep. 11, 2009

Related U.S. Application Data
(60) Provisional application No. 61/191,717, filed on Sep. 11, 2008.
(60) Provisional application No. 61/192,557, filed on Sep. 19, 2008.
(60) Provisional application No. 61/192,558, filed on Sep. 19, 2008.
(60) Provisional application No. 61/192,560, filed on Sep. 19, 2008.

(51) Int. Cl. G06Q 50/00; A61B 5/00; G06Q 10/00; G06T 11/20

(52) U.S. Cl. 705/3; 600/300; 345/440.2

(57) ABSTRACT

A method for preparing a medical report. Some example methods include receiving one or more medical data readings, where respective individual medical data readings include a numerical result of a medical test; processing the medical data readings into report data, where the report data includes the individual medical data readings and categorized ranges associated with the medical tests associated with individual medical data readings; and creating a medical data report including the report data, graphical representations of individual medical data readings and the respective categorized ranges, and textual descriptive information pertaining to the respective medical data readings.

### CROSS REFERENCE TO RELATED APPLICATIONS

This application claims the benefit of U.S. Provisional Application No. 61/191,717, filed Sep. 11, 2008, U.S. Provisional Application No. 61/192,557, filed Sep. 19, 2008, U.S. Provisional Application No. 61/192,558, filed Sep. 19, 2008, and U.S. Provisional Application No. 61/192,560, filed Sep. 19, 2008, all of which are incorporated by reference.

### COPYRIGHT NOTICE

A portion of the disclosure of this patent document contains material which is subject to copyright protection. The copyright owner has no objection to the facsimile reproduction by any-one of the patent document or the patent disclosure, as it appears in the Patent and Trademark Office patent file or records, but otherwise reserves all copyright rights whatsoever.

### BACKGROUND

The present disclosure is directed, inter alia, to methods of knowledge generation, individual bioprofile generation, information assembly, individual bioprofile scoring, and medical data report preparation.

### SUMMARY

Exemplary embodiments may include a method for preparing a medical report. Some example methods may include receiving one or more medical data readings, where respective individual medical data readings include a numerical result of a medical test; processing the medical data readings into report data, where the report data includes the individual medical data readings and categorized ranges associated with the medical tests associated with individual medical data readings; and creating a medical data report including the report data, graphical representations of individual medical data readings and the respective categorized ranges, and textual descriptive information pertaining to the respective medical data readings.

In an aspect, a method for preparing a medical data report may include receiving one or more medical data readings, where respective individual medical data readings include a numerical result of a medical test; processing the medical data readings into report data, where the report data includes the individual medical data readings and categorized ranges associated with the medical tests associated with individual medical data readings; and creating a medical data report including the report data, graphical representations of individual medical data readings and the respective categorized ranges, and textual descriptive information pertaining to the respective medical data readings.

In a detailed embodiment, creating the medical data report may include producing a tangible report for presentation to a patient associated with the medical data readings.

In a detailed embodiment, the categorized ranges associated with individual medical tests may include at least two of poor, good, and excellent. In a detailed embodiment, the graphical representations may include distinguishable colors associated with each of the categorized ranges, respectively.

In a detailed embodiment, the graphical representations may depict the medical data readings on respective categorized ranges, wherein at least one of the categorized ranges includes a high value and/or a low value. In a detailed embodiment, the textual descriptive information may include at least one of a description of a significance of a high reading or a low reading, a suggested action for causing a change in the respective medical data reading, and a suggestion to discuss the respective medical data reading with a medical professional. In a detailed embodiment, the report may include a health summary page including numbers of readings falling within individual categorized ranges.

In a detailed embodiment, the report may include a detailed health summary page including a bar graph representation of individual medical data readings. In a detailed embodiment, the detailed health summary page may include instructions pertaining to interpretation of the report.

In a detailed embodiment, the report data, the graphical representations of individual medical data readings and the respective categorized ranges, and the textual descriptive information pertaining to the respective medical data readings may be provided on at least one readings page.

In a detailed embodiment, the graphical representations of the individual medical data readings may be depicted using a graphical scale. In a detailed embodiment, the graphical scale may include a bar including medical data indicia along the bar and a representation of at least one of the individual readings also indicated along the bar. In a detailed embodiment, the graphical scale may include a balance including a first end representing a normal reading and a second end representing a measured reading. In a detailed embodiment, the balance may be tilted towards a greater of the normal reading and the measured reading.

In an aspect, a method of communicating medical data to a patient may include processing medical test data into report data, where the report data includes individual readings and categorized ranges associated with individual medical tests; and creating a tangible report that includes, for each medical test, (1) a graphical display of the respective individual reading and the associated categorized ranges and (2) a text description providing information pertaining to the medical test.

In a detailed embodiment, the graphical display may include individual colors associated with the categorized ranges.

In a detailed embodiment, the information pertaining to the medical test may include advice for improving the respective individual reading. In a detailed embodiment, the advice may include diet advice.

In a detailed embodiment, the report may include a listing of a number of readings associated with individual categories associated with the categorized ranges. In a detailed embodiment, the report may include a pie chart illustrating relative numbers of readings associated with each of the individual categories.

In a detailed embodiment, the report may include, for individual readings, a bar graph representation of a category associated with the categorized ranges.

In a detailed embodiment, the information pertaining to the medical test may include advice suggesting consultation with a medical professional.

In a detailed embodiment, the report may include a listing of a priority subset of the readings; and wherein the priority subset includes a plurality of readings for which action may be most important. In a detailed embodiment, the listing of the priority subset of the readings may include an explanation of each of the individual readings comprising the priority subset.

In a detailed embodiment, the individual medical tests may include molecular analysis of a biological sample for analytes comprising a molecular bioprofile. In a detailed embodiment, the molecular bioprofile may be produced by identifying a set of relevant bioindicators; correlating the set of relevant bioindicators to create a network; and weighting each of the relevant bioindicators in the set according to its importance. In a detailed embodiment, molecular analysis of the biological sample may include mass spectrometry of a blood sample. In a detailed embodiment, the molecular analysis of the biological sample may include hematologic analysis of a blood sample.

### BRIEF DESCRIPTION OF THE DRAWINGS

The detailed description refers to the following figures in which:

FIG. 1 is a diagram showing a general overview of an exemplary Knowledge Generator;

FIG. 2 is a diagram showing an exemplary Knowledge Generator that produces a health and wellness assessment report based upon analysis of a patient's blood sample;

FIG. 3 is a diagram showing an exemplary Knowledge Generator that produces a knowledge report based upon environmental information and data in the context of barometric pressure and bass fishing;

FIG. 4 is a schematic diagram of an exemplary computing system which may be used to perform exemplary methods according to the present disclosure;

FIG. 5 is a diagram showing an overview of an exemplary platform for performing methods described herein;

FIG. 6 is a flow diagram of an exemplary method for creating a bioprofile;

FIG. 7 is a flow chart showing an exemplary process of obtaining and analyzing a sample and reporting the results of the analysis;

FIG. 8 is a diagram showing an overview of an exemplary Iterative Enrichment process;

FIG. 9 is a diagram showing an overview of an exemplary search list generation process;

FIG. 10 is a diagram showing an overview of an exemplary process including insertion of the search list into a biological analysis;

FIG. 11 is a flowchart showing an exemplary process for scoring a molecular bioprofile;

FIG. 12 is an exemplary plot of score versus measurement;

FIG. 13 is an exemplary pie chart showing the accumulated priority and the number of molecules falling into normal and abnormal ranges;

FIG. 14 illustrates an example health summary page;

FIG. 15 illustrates an example detailed health summary page;

FIG. 16 illustrates an example readings page;

FIG. 17 illustrates an example summary page;

FIG. 18 illustrates an example cardiovascular summary page;

FIG. 19 illustrates an example cardiovascular bioprofile page;

FIG. 20 illustrates an example cardiovascular out of balance readings page;

FIG. 21 illustrates an example cardiovascular in balance readings page;

FIG. 22 illustrates an example cardiovascular resources page; and

FIG. 23 illustrates an example workplace report; all in accordance with at least some aspects of the present disclosure.

### DETAILED DESCRIPTION

The present disclosure is directed to, inter alia, methods of knowledge generation, individual bioprofile generation, information assembly, individual bioprofile scoring, and medical data report preparation.

Method of Knowledge Generation

This portion of the disclosure relates generally to the integration of measurement and knowledge and information assembly and the organization of the resulting platform outputs resulting in specific new knowledge generation. More specifically, it relates the integration and combination of data outputs from analytical instrumentation with knowledge assembly methods resulting in a specific output organized with various information assembly techniques.

The present disclosure contemplates that, traditionally, platforms have been based exclusively on analytical measurements of very specific biological samples. This approach restricts the flexibility and relevance of the outputs of the platform in knowledge generation.

The present disclosure contemplates that knowledge and information assembly methods, such as text mining and pathway and network analysis have proven effective to generate limited knowledge about a specific system. On the other hand, measurements through analytical instrumentation have been effective at quantifying the amounts of some entity within a specific biological sample. However, these two methods, when used exclusively, have provided limited new knowledge generation capabilities.

This portion of the disclosure describes exemplary embodiments providing integration of analytical and knowledge and information assembly methods. This allows for the expansion of the flexibility and effectiveness of each component part of a non-linear platform. It also allows for the optimization of the resulting outputs from the platform. Exemplary methods provide comprehensive, correlated, relevant and outputs through the integration of the analytics and knowledge/information assembly. Additionally, exemplary methods also allow for targeted platform outputs. An exemplary embodiment is able to generate a list of molecules and bioprofiles from up-to-date literature (through text mining, for example) and through demographic studies (or other analyses) to define and describe a specific health state with a human system; and then compare the analysis of a patient's tissue sample against the list of molecules and/or bioprofiles so that the patient's health state can be assessed. In such exemplary embodiment, a plurality of molecules are chosen to be measured from the patient's tissue sample based upon the results of the knowledge/information assembly, where such molecules were chosen for measurement because the knowledge/information assembly process indicated that such molecules are indicators of a particular area of health. From the tissue sample, then, each of the plurality of molecules were measured and scored based upon three primary criteria: (1) the impact that health condition has on that particular molecule; (2) the patient's molecular score compared to a general population of “healthy” individuals (comparison of molecular measurement versus demographic data); and (3) the amount of scientific evidence supporting the impact of that particular health condition on that molecule (text mining to determine relevance of particular molecule to particular health condition).

Exemplary embodiments include an integrated platform that is able to accept any type of biological sample from human, animal, plant, or environmental systems, for example. An exemplary method includes the utilization of a knowledge assembly process, such as text mining, to direct the processing of the contents subjected to the analytical instrumentation. There are no requirements for a specific analytical instrumentation in this process. The data outputs from the analytical instrumentation, based on the knowledge assembly process, are then manipulated using various information assembly methods resulting in the direct output of the platform.

Utilizing information assembly methods, exemplary platforms generate various outputs. For example, an exemplary platform is able to generate graphical representations of the data outputs from the analytical instrumentation. Along with graphical representation, the analytical instrumentation data outputs are able to be encompassed with the most up-to-date literature or the platform is able to generate a list of correlated and relevant molecules describing any biological state or system. As an example, an exemplary embodiment is able to generate a list of molecules, bioprofiles, and up-to-date literature to define and describe a specific health state with a human system.

Exemplary embodiments are also flexible by allowing simple variations in the process flow. For instance, knowledge assembly, while directing measurement objectives, can generate in-silico data without any experimental data. These in-silico outputs can then proceed through the platform. The database is also flexible because any output from any stage of the platform can be stored at any time. The data is then able to be used any time at any step in the process flow.

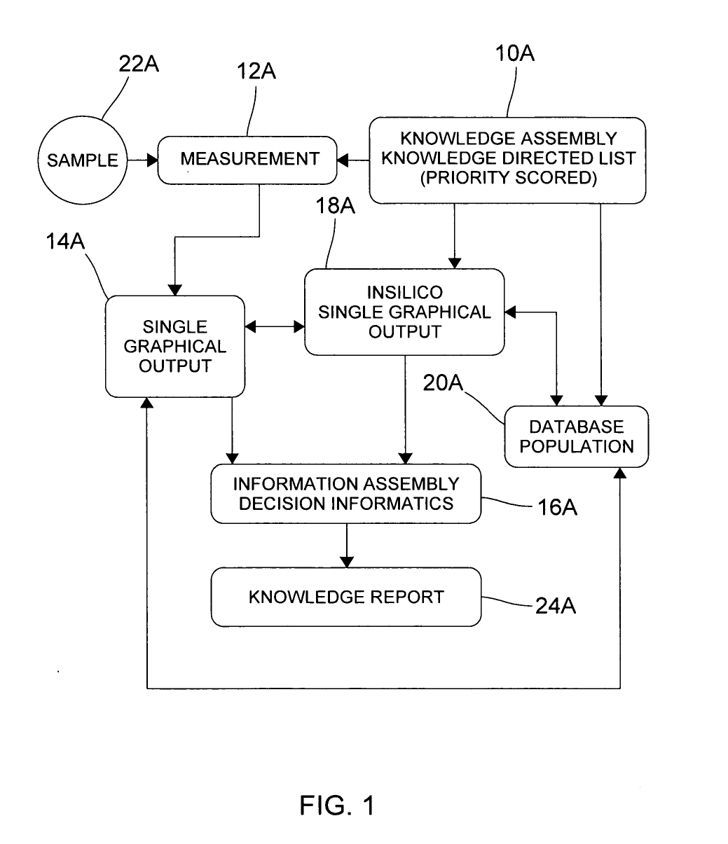

As shown in FIG. 1, an exemplary method of generating knowledge includes knowledge assembly 10A, sample measurement 12A, production of a single graphical output 14A, and information assembly 16A. Knowledge assembly 10A directs the measurements and generates in-silico graphical outputs 18A, as well as providing database 20A iterative enrichment and storage. Measurement 12A includes insertion of an experimental sample 22A and generation of a graphical output 14A. The graphical output 14A is stored in database 20A and allows for complementary in-silico graphic development 18A, and is subjected to information assembly 16A. Information assembly includes generation of a knowledge report 24A (which may be in the form of a hard-copy or electronic report).

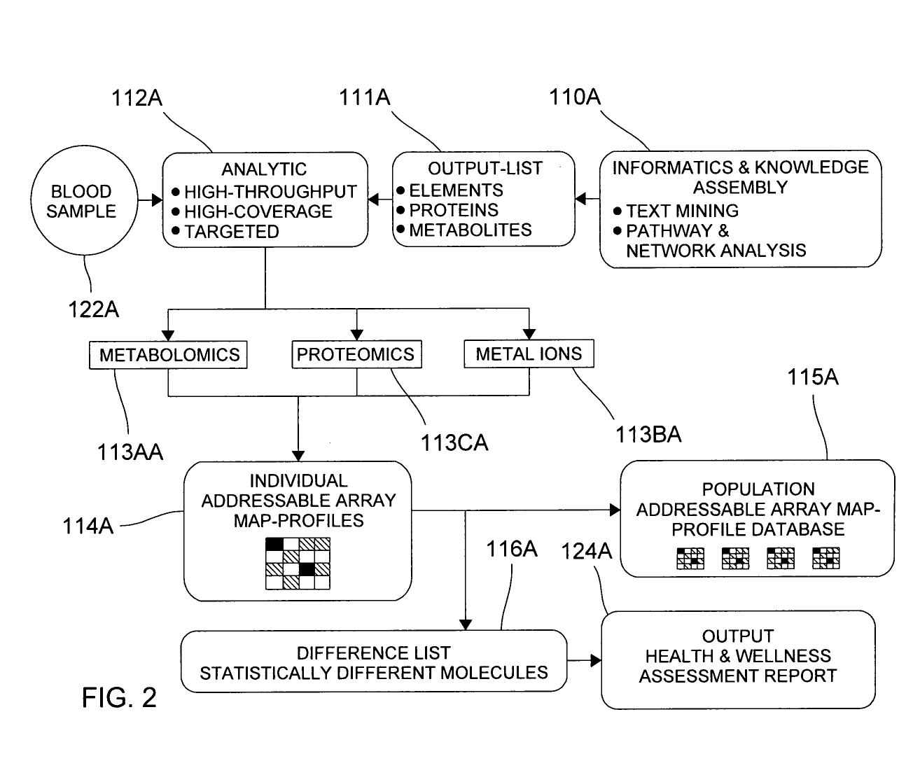

FIG. 2 illustrates an exemplary platform for implementing a knowledge generation method. Knowledge assembly 110A includes development of analytical objectives and identification of material to be subjected to instrumentation using text mining and network and pathway analysis. The outputs 111A of the knowledge assembly are stored in a platform database and include, for example, specific elements, proteins, and metabolites. A sample, such as a blood sample 122A, is provided to analytical instrumentation 112A (such as mass spectrometry, immunoassay, spectrophotometric assay, etc.), which analyzes the sample 122A in comparison with the results 111A of the knowledge assembly 110A and generates data output 113AA, 113BA, 113CA, which are stored in the platform database. In this example, the data outputs 113AA, 113BA, 113CA relate to metabolomics 113AA, metal ions 113BA, and proteomics 113CA. The analytical instrumentation 112A is designed to be high-throughput, high-coverage, and targeted. The outputs 113A, 113BA, 113CA are used to create a graphical representation and a bioprofile 114A. Information assembly 116A includes producing a difference list of statistically different molecule measurements (as directed by knowledge assembly 111A and in comparison to population/demographic data 115A) and organization of the platform output 124A and a computer program/process. Generation of the difference list utilizes the population addressable array map profile database 115A.

In general, an exemplary method begins with a knowledge assembly process 110A. For example, a text mining process is used in conjunction with a biological pathway analysis. This initial process directs and hones the subject matter that will be analyzed using the analytical instrumentation 112A. For example, the output from the knowledge assembly process 110A could be a list of molecules to be analyzed by mass spectrometry (MS) instrumentation 112A. Upon analyzing the set of molecules from the knowledge assembly process 110A, the instrument 110A will produce a set of data outputs 113AA, 113BA, 113CA. These outputs 113AA, 113BA, 113CA are then subjected to various information assembly methods in order to generate a graphical representation 114A and various related literature. For example, the integrated platform produces a graphical comparison 116A of an individual versus a population specific to the analyzed molecules. In some embodiments, the platform may also produce literature presenting a bioprofile of a specific human area of health and wellness 124A.

An exemplary knowledge assembly process 110A begins with text mining. As will be appreciated by those of ordinary skill in the art, there are numerous text mining and semantic analysis algorithms available for use with these or other embodiments described herein; all of which are within the scope of the present disclosure. An exemplary text mining approach rapidly identifies target molecule candidates from the corpus of data publicly available via PubMed. Using a simple set of search terms, a list of abstracts is retrieved from PubMed. This list is reduced to only the most relevant abstracts by a sophisticated ranking system that scores a list of phrases or terms of interest. The ranking system takes into account syntactic and semantic information to create a relevancy score. Candidate target molecules are then chosen from a list of molecules associated with the documents having the highest overall relevancy score. Using this approach, the method takes advantage of the wealth of knowledge generated from more than 50 years of biological research to determine what species should be targeted for analysis.

More specifically, an exemplary knowledge assembly process 110A may begin with development of a word or list of words that describe a disease or condition that is relevant. For example, a word that can be used as a descriptor for the overall condition of stress is “stress.” Through search approaches, the descriptor “stress” is interrogated against all of available information space (such as PubMed). The outcome of this process is a library of abstracts and manuscripts that contain the descriptor word “stress.” To further the process, the library of abstracts and manuscripts are interrogated against a list of 10,000,000 known molecules, for example. The two searches connect “stress” to a group of molecules. The end product is a library of abstracts and manuscripts that contain the descriptor (stress) and connected molecules. The molecules connected to stress, in the end library of abstracts and manuscripts, are scored to provide a list. Each time a molecule appears in an abstract or manuscript it is scored. For example, a common molecule connected to stress is cortisol. Each time cortisol appears in information space it receives a unit of score. The end product is a weighted list of molecules 111A. The ranking or weighting factor within the list indicates the frequency and type of connection between the molecule and the descriptor.

In another example, Iterative Enrichment begins with a search of an article/abstract database, such as the PubMed database. PubMed provides access to decades of medical research that can be mined to provide biological context to a targeted measurement approach. In this exemplary embodiment, the PubMed repository is first searched using a descriptor of health and wellness expression (e.g., Type II Diabetes). The query results in a candidate set of abstracts which are subjected to further text mining. Using a more refined list of expressions, the candidate set of abstracts is ranked based upon the content's health and wellness significance. The significance is determined by computing the overall rank of the article or abstract based upon each of the individual expressions. The algorithm used to rank individual expressions utilizes modern techniques like full-text indexing, noise word removal, word stemming, and synonyms substitution, thereby producing a high quality rank based on syntactic and semantic relevance.

The ranked abstracts are then searched again for the presence of molecular species as defined by the PubMed list of chemical entities (1.0×10⁷). For example, the ranked abstracts are searched for proteins, metabolites, and essential nutrients. If a molecule (or element) is observed anywhere in the abstract, it is entered into the molecular list. A simple scoring scheme generates a ranking of the molecules in the list. The scoring scheme considers the molecular entity's frequency of occurrence and the score associated with the ranked abstract. The score for each molecule represents the sum of these scores.

Conventionally, such searching efforts have created no significant arguable biological context. To overcome this, in exemplary embodiments, the most relevant molecules from the search generated list are subjected to a biological analysis, such as a pathway/network analysis. For example, the top 50% of the ranked molecules may be interrogated against a pathway/network analysis. The molecules are inserted into pathway/network software, generating biological context to the search-generated list. The end products are a pictorial representation of the networks associated with the molecules inserted into the program. Also, a new weighted pathway/network list of molecules is generated based on the pathway/network analysis. An example of an available pathway and network analysis tool is the Ingenuity® Pathway Analysis products provided by Ingenuity Systems.

More specifically, in exemplary embodiments, determinations of molecular correlations can be performed using molecular bioprofiling data. Such measurements are beneficial in that the correlation or combinations of correlations help determine the overall status of an individual's health. An exemplary molecular correlation network application combines interactive visualization and statistical data mining. Interactive visual data mining (IVDM) is a human driven mining approach that uses visualization and interaction. It attempts to extract useful and potentially unsuspected patterns from data sets. Rather than using the data to derive certain information based on an a priori human knowledge structure, IVDM accommodates novel data mining goals and holds great potential for systems biology. Exemplary methods may minimize the necessity for communication between bioinformaticians and biologists during molecular correlation network analyses.

Exemplary software for molecular correlation network studies automatically integrates molecular expression data generated from a proteomic, metabolomic, and metalomic platforms; interactively analyzes intermolecular correlations using different statistical models; and performs interactive visual analysis of molecular profiles in time course studies. Data inputs can be stored in various databases including Access, PostgreSQL and MySQL; or data files, such as text or Excel files.

Exemplary software for molecular correlation includes three modules: data management, scientific computation, and interactive visualization. The data management module connects data from the various databases and files. It also communicates with the scientific computation module to obtain the intermediate computational results. The scientific computation module includes a library of scientific computation algorithms. Computation of correlation and data model fitting is done by the scientific computation module. The interactive visualization module serves as the core of the system. It takes information from the data management and scientific computation modules and provides interactive visualization on the computer screen, for example.

Exemplary software implements both parametric and non-parametric pair-wise measures of molecular correlation, including the parametric Pearson product-moment correlation (rₚ), the non-parametric Spearman correlation (rₛ), and the non-parametric Kendall's coefficient of rank correlation (τ).

In an exemplary embodiment, the expanded molecular lists are ranked using a scoring scheme based on: subnetwork score, molecular connectivity within a subnetwork (typically few molecules are observed in multiple subnetworks), and the biological functions in which the molecules are known to play a role. In addition, the frequency of occurrence is used as a scoring parameter. As a result, scores are additive for molecules observed in more than one subnetwork. A more detailed description of an exemplary scoring scheme follows.

The subnetwork score is related to the p-value calculated for the subnetwork (−log p). This score is then normalized to a value of 10 across all molecules for a given Health and Wellness List.

The molecular connectivity is calculated based on the total number of direct and indirect regulation relationships that are observed for each molecule. Each of the former and latter relationships is assigned a score of 1 and 0.5, respectively. These are summed together to effectively determine network hubs.

Biological functions assigned to the network by the pathway and network analysis software are vetted for their relevance to the respective Health and Wellness List. Biological functions scores (scale of 1 to 10) are then assigned to the appropriate molecules (i.e., those that play a role in the particular function).

The final score for each molecule is tabulated as the sum of the subnetwork, relationship, and biological functions score.

In an exemplary embodiment, approximately 50 to 100 seed terms from the top scoring molecules in the text mining list are selected for biological network and pathway analysis. Molecules are selected based on their text mining score, their association with the physiological state (Type II Diabetes, for example), and their ability to be used by the pathway and network analysis software in a biological network analysis. Molecular families which do not have single species represented in the network and pathway analysis are excluded from the molecular seed file. The seed molecules are imported into the pathway and network analysis software and a network analysis performed. All of biomolecular pathway space is searched to create a biological condition network of related molecules. An exemplary biological network may illustrate direct regulation of one molecule by another (direct contact) and indirect regulation. Self regulation may also be indicated (direct and indirect).

The connectivity of elements in the networks is related to their regulation of each other, both direct and indirect regulation. Each defined intermolecular regulation is assigned a p-value from which a subnetwork p-value is calculated. In an embodiment, this is limited to 35 molecules by the pathway and network analysis software to enhance visualization. This value represents the probability of the accumulated molecules correlating to a random grouping. To obtain the best subnetworks (those of highest p-value), new molecules are incorporated (i.e., those not included in the original seed molecule list). Thus, after one network analysis, the list of potential target molecules increases significantly. In an exemplary embodiment, three iterations of network analysis are used to augment the target list with molecules associated with specific physiological conditions.

In an exemplary embodiment, the standard flow of information through iterative enrichment (IE) begins with a search to discover any mathematical associations. The mathematical associations are then subjected to biological analysis, such as a pathway/network analysis, to provide the biological context. However, the information processed through the iterative enrichment process has no standard operation protocol. Any type of information, from any stage of the IE process, is able to permeate into the overall process at any point and proceed from that point unhindered.

As an example, information is subjected to the IE process and stored in an internal database. That information, in the future, could be withdrawn from the database and subjected to an iterative search of all new available information space. The information would then proceed through the IE process for the second time. This chain of events is able to proceed an infinite number of times. During each round, the information will be enriched through the iterative cycles.

Internal platform database information can be isolated, searched and interrogated to determine any mathematical associations within the data. The mathematical associations are then subjected to biological analysis to provide biological context.

As described above, exemplary embodiments include a process known as Iterative Enrichment in which knowledge assembly tools 110A are used to create an output list 111A of scored (weighted) molecules and elements (primarily metal ions) to be targeted for profile comparisons that determine individual health and wellness. The list is obtained through extensive text mining as well as pathway and network analysis.

Next, in an exemplary process, an individual blood sample is analyzed using high-throughput analytical instrumentation 112A that provides efficient, targeted coverage, and characterization of complex biological samples 122A. Mass informatics and bioinformatics are then used to produce an Addressable Array Map 114A (AAM) for the individual using the datasets obtained from the different measurements. In order to create an AAM 114A, specific physical properties (e.g., molecular weight, HPLC (high performance liquid chromatography) retention time) for individual molecules and elements are transformed via a multi-dimensional projection algorithm onto a grid of discrete coordinates. The resulting AAM 114A is then used in a comparative analysis of individuals against defined populations. The final output is a differential list 116A of molecules with statistically significant differences in concentration between the individual AAM 114A and the population AAMs 115A. This output is known as a molecular bioprofile. The delta (Δ) list 116A is read into the knowledge assembly module to ascertain the relative health and wellness of the individual.

The output of the exemplary integrated platform approach using Iterative Enrichment and high-throughput analyses is a molecular bioprofile 116A. This is defined as a biologically related network of bioindicators that determine the biological health and wellness status of the individual compared to a relevant control population. It should be noted that a bioindicator differs from a conventional biomarker. A bioindicator has both defined biological relevance to the health condition as well as correlation to other bioindicators, as it pertains to the health condition under scrutiny.

A knowledge based text mining search and pathway/network analysis identifies relevant bioindicators (more than 20, for example) for the health condition being scrutinized. The individual bioindicators are then correlated to create a biologically relevant network and each scored according to the importance of its role in the network. Each bioindicator is also quantified in the biological fluid being measured and compared to normal, healthy ranges for the same analyte. The normal, healthy ranges are included in a population map 115A that is produced by generating in silico population data and also through the accumulation of biological samples. These weighted, correlated and quantified bioindicators form a molecular bioprofile.

The importance of the molecular bioprofile is that it identifies and establishes each analyte's biological function in the biologically relevant network associated with each health condition being evaluated. This has significant potential in both predictive and preventive medicine.

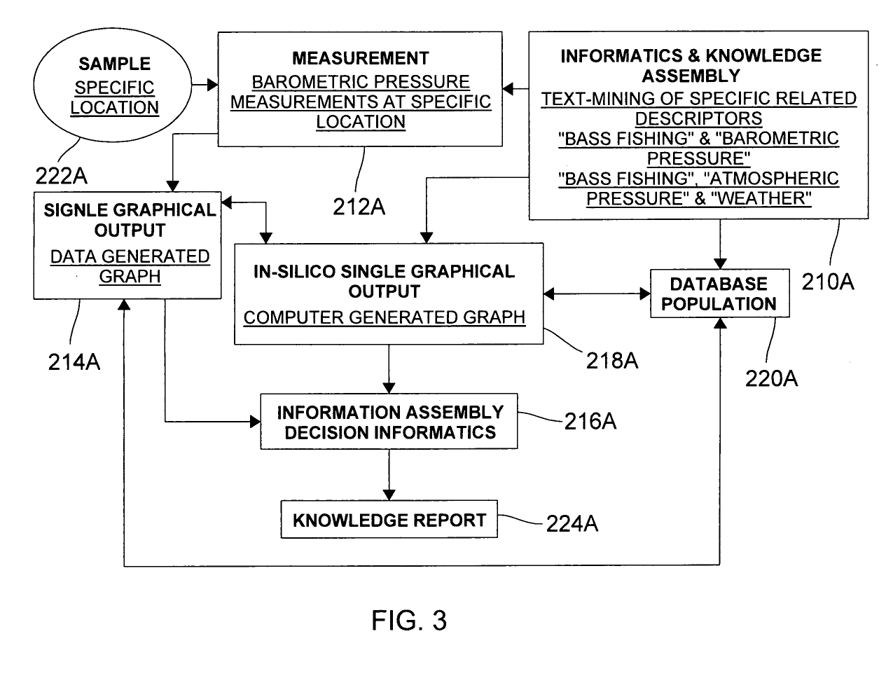

In an alternative exemplary embodiment depicted in FIG. 3 (to provide an example of how embodiments can be utilized outside of healthcare and wellness assessments), a knowledge generation process is applied in the context of barometric pressure in bass fishing. An informatics and knowledge assembly process 210A includes text mining using descriptors, such as “bass fishing,” “barometric pressure,” “atmospheric pressure,” and “weather.” In this example, the sample 222A is a specific location. The measurement 212A includes the barometric pressure at the specific location. The single graphical output 214A includes a data generated graph. The single graphical output 214A is provided to the information assembly decision informatics 216A, which produces a knowledge report 224A. In addition, the in-silico single graphical output 218A produces a computer generated graph. As shown, various components are connected to the database population 220A.

Although exemplary embodiments have been described above as utilizing the PubMed database and analyzing data in a human health context, it is within the scope of this disclosure to utilize the methods and processes herein to analyze information and data in any context. For example, one or more corpora in any subject area may be mined. Additionally, the knowledge assembly process can begin with any information related to the analysis. For example, in the health-care model, the knowledge assembly process can begin with any possible patient information, such as information ranging from a single sign or symptom to a completely diagnosed disease.

Exemplary platforms are capable of seamlessly integrating any instrumentation that results in a data output. These data outputs cover all of data output space. For example, exemplary platforms may integrate data including any data ranging from vital signs to genomics in a healthcare application. In another exemplary application, such as a weather application, it is possible to integrate data from any meteorological instrumentation. Generally, any instrumentation that results in a data output can be utilized by exemplary platforms.

An exemplary method of generating knowledge may include assembling knowledge by searching in at least one corpus and identifying at least one relevant quantitative parameter; measuring the quantitative parameter in a sample; producing an output related to the quantitative parameter; assembling information by comparing the output to a control value; and generating a knowledge report including the information.

An exemplary method of analyzing a sample may include identifying a plurality of molecules for analysis; correlating the plurality of molecules based on at least one of a biological function and an importance of each of the plurality of molecules; analyzing a sample to measure a concentration of each of the plurality of molecules; comparing the measured concentrations of each of the plurality of molecules to respective expected concentrations of each of the plurality of molecules; and generating a list including each of the plurality of molecules for which the measured concentration was statistically different from the expected concentration.

Computer-Executed Methods

Exemplary methods according to the present disclosure may be implemented in the general context of computer-executable instructions that may run on one or more computers, and exemplary methods may also be implemented in combination with program modules and/or as a combination of hardware and software. Generally, program modules include routines, programs, components, data structures, etc., that perform particular tasks or implement particular abstract data types. Moreover, those skilled in the art will appreciate that exemplary methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices. Exemplary methods may also be practiced in distributed computing environments where certain tasks are performed by remote processing devices that are linked through a communications network. In a distributed computing environment, program modules can be located in both local and remote memory storage devices.

An exemplary computer typically includes a variety of computer readable media. Computer readable media can be any available media that can be accessed by the computer and includes volatile and non-volatile media, removable and non-removable media. By way of example, and not limitation, computer-readable media can comprise computer storage media and communication media. Computer storage media includes volatile and non-volatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer.

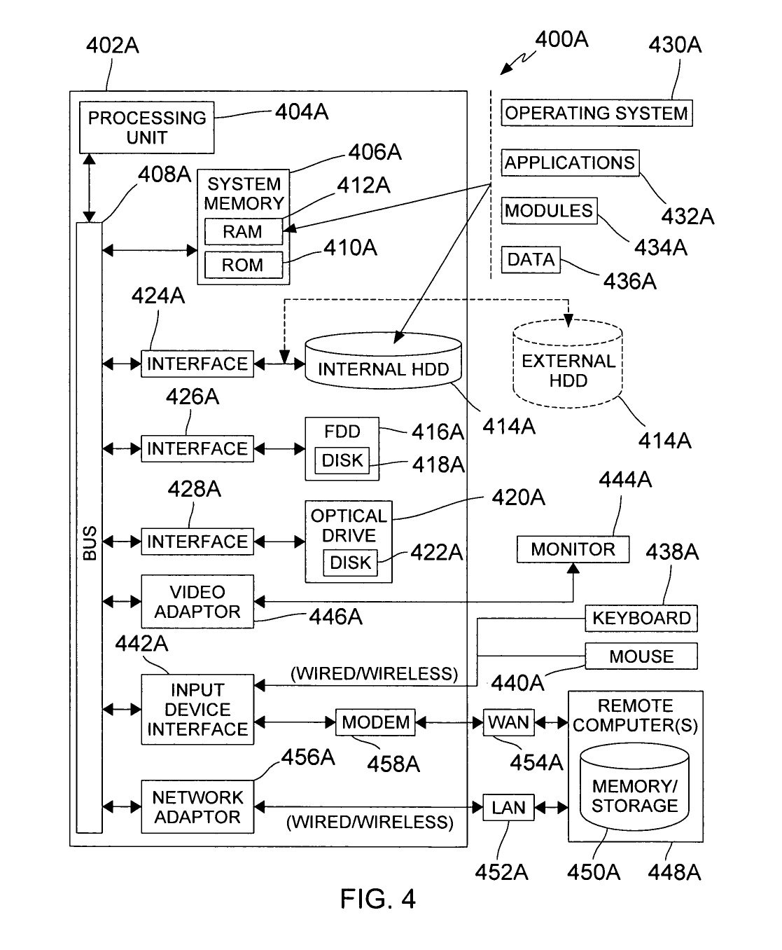

With reference to FIG. 4, an exemplary computing system 400A includes a computer 402A including a processing unit 404A, a system memory 406A, and a system bus 408A. The system bus 408A provides an interface for system components including, but not limited to, the system memory 406A to the processing unit 404A. The processing unit 404A can be any of various commercially available processors, for example. Dual microprocessors and other multi processor architectures may also be employed as the processing unit 404A. The system bus 408A can be any of several types of bus structure that may further interconnect to a memory bus (with or without a memory controller), a peripheral bus, and a local bus using any of a variety of commercially available bus architectures. The system memory 406A includes read-only memory (ROM) 410A and random access memory (RAM) 412A. A basic input/output system (BIOS) is stored in a non-volatile memory 410A such as ROM, EPROM, EEPROM, which BIOS contains the basic routines that help to transfer information between elements within the computer 402A, such as during start-up. The RAM 412A can also include a high-speed RAM such as static RAM for caching data.

The computer 402A further includes an internal hard disk drive (HDD) 414A (e.g., EIDE, SATA), which internal hard disk drive 414A may also be configured for external use in a suitable chassis (not shown), a magnetic floppy disk drive (FDD) 416A, (e.g., to read from or write to a removable diskette 418A) and an optical disk drive 420A, (e.g., reading a CD-ROM disk 422A or, to read from or write to other high capacity optical media such as the DVD). The hard disk drive 414A, magnetic disk drive 416A and optical disk drive 420A can be connected to the system bus 408A by a hard disk drive interface 424A, a magnetic disk drive interface 426A and an optical drive interface 428A, respectively. The interface 424A for external drive implementations includes at least one or both of Universal Serial Bus (USB) and IEEE 1394 interface technologies.

The drives and their associated computer-readable media provide nonvolatile storage of data, data structures, computer-executable instructions, and so forth. For the computer 402A, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing novel methods of the disclosed architecture.

A number of program modules can be stored in the drives and RAM 412A, including an operating system 430A, one or more application programs 432A, other program modules 434A and program data 436A. All or portions of the operating system, applications, modules, and/or data can also be cached in the RAM 412A. It is to be appreciated that the disclosed architecture can be implemented with various commercially available operating systems or combinations of operating systems.

A user can enter commands and information into the computer 402A through one or more wire/wireless input devices, for example, a keyboard 438A and a pointing device, such as a mouse 440A. Other input devices (not shown) may include a microphone, an IR remote control, a joystick, a game pad, a stylus pen, touch screen, or the like. These and other input devices are often connected to the processing unit 404A through an input device interface 442A that is coupled to the system bus 408A, but can be connected by other interfaces, such as a parallel port, an IEEE 1394 serial port, a game port, a USB port, an IR interface, etc.

A monitor 444A or other type of display device is also connected to the system bus 408A via an interface, such as a video adapter 446A. In addition to the monitor 444A, a computer typically includes other peripheral output devices (not shown), such as speakers, printers, etc.

The computer 402A may operate in a networked environment using logical connections via wire and/or wireless communications to one or more remote computers, such as a remote computer(s) 448A. The remote computer(s) 448A can be a workstation, a server computer, a router, a personal computer, portable computer, microprocessor-based entertainment appliance, a peer device or other common network node, and typically includes many or all of the elements described relative to the computer 402A, although, for purposes of brevity, only a memory/storage device 450A is illustrated. The logical connections depicted include wire/wireless connectivity to a local area network (LAN) 452A and/or larger networks, for example, a wide area network (WAN) 454A. Such LAN and WAN networking environments are commonplace in offices and companies, and facilitate enterprise-wide computer networks, such as intranets, all of which may connect to a global communications network, for example, the Internet.

When used in a LAN networking environment, the computer 402A is connected to the local network 452A through a wire and/or wireless communication network interface or adapter 456A. The adaptor 456A may facilitate wire or wireless communication to the LAN 452A, which may also include a wireless access point disposed thereon for communicating with the wireless adaptor 456A. When used in a WAN networking environment, the computer 402A can include a modem 458A, or is connected to a communications server on the WAN 454A, or has other means for establishing communications over the WAN 454A, such as by way of the Internet. The modem 458A, which can be internal or external and a wire and/or wireless device, is connected to the system bus 408A via the serial port interface 442A. In a networked environment, program modules depicted relative to the computer 402A, or portions thereof, can be stored in the remote memory/storage device 450A. It will be appreciated that the network connections shown are exemplary and other means of establishing a communications link between the computers can be used.

The computer 402A is operable to communicate with any wireless devices or entities operatively disposed in wireless communication, for example, a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices. Wi-Fi, or Wireless Fidelity, allows connection to the Internet from a couch at home, a bed in a hotel room, or a conference room at work, without wires. Wi-Fi is a wireless technology similar to that used in a cell phone that enables such devices, for example, computers, to send and receive data indoors and out; anywhere within the range of a base station. Wi-Fi networks use radio technologies called IEEE 802.11x (a, b, g, etc.) to provide secure, reliable, fast wireless connectivity. A Wi-Fi network can be used to connect computers to each other, to the Internet, and to wired networks (which use IEEE 802.3 or Ethernet).

Method of Generating an Individual Bioprofile

This portion of the disclosure relates generally to the creation of unique molecular bioprofiles for individuals for the measurement of health, wellness, and disease. More specifically, it relates to the creation of unique networks that contain specific biological content enabling prediction of the health, wellness, and disease status of an individual.

The present disclosure contemplates that, historically, health has been defined by the absence or presence of disease. This has led to the current clinical diagnostics sector “single diagnostic marker/disease” model used to determine a person's overall ill-health. More recently, some fledgling attempts have been made to demonstrate the use of biomarker panels (typically 3-10 analytes) as indicators of disease presence and progression. An example is the wide use of creatine kinase-MB, troponin C, and C-Reactive protein in clinical situations to diagnosis myocardial infarction. In this case, it is important to note that the individual components are used in an additive manner to increase the specificity and sensitivity of the diagnosis.

The present disclosure contemplates that the identification and development of new, more specific and sensitive biomarkers of health or disease has been remarkably slow in development. This is due in part to the linear, one dimensional approach adopted by most biomarker discovery programs. In this process, 'omics (a field of study in biology ending in the suffix -omics, such as genomics or proteomics) data from a control sample cohort is compared to that obtained from a disease sample cohort. Differences in concentrations of specific analytes from different cohorts are considered indicative of biomarker candidates. Since there are often hundreds of analytes that differ between the two cohorts, and no biological function has been ascribed to each putative biomarker, this is akin to “looking for a needle in a haystack.”

In contrast to other clinical diagnostics and biomarker discovery programs, the present disclosure describes the use of a molecular bioprofile of a specific condition in human health, wellness, and disease. An exemplary molecular bioprofile includes a network of 20+ biologically correlated and relevant molecules that determines the biological state of a human health condition compared to a control population. It is within the scope of the disclosure to utilize a network of many more or potentially fewer than 20 molecules.

The output of exemplary platforms provides broad information and knowledge about complex physiology events. Exemplary integrated platforms use informatics and knowledge assembly to target physiologically relevant analytes for analysis. Targeted analytes (including, for example, metal ions/elements, proteins, and metabolites in human biological fluids) are measured using high-throughput, multi-dimensional instrumentation. For example, samples may be analyzed using mass spectrometry. The targeted approach can be performed on a complex biological fluid, such as plasma. In comparison, conventional approaches are not targeted for this type of analysis and processing.

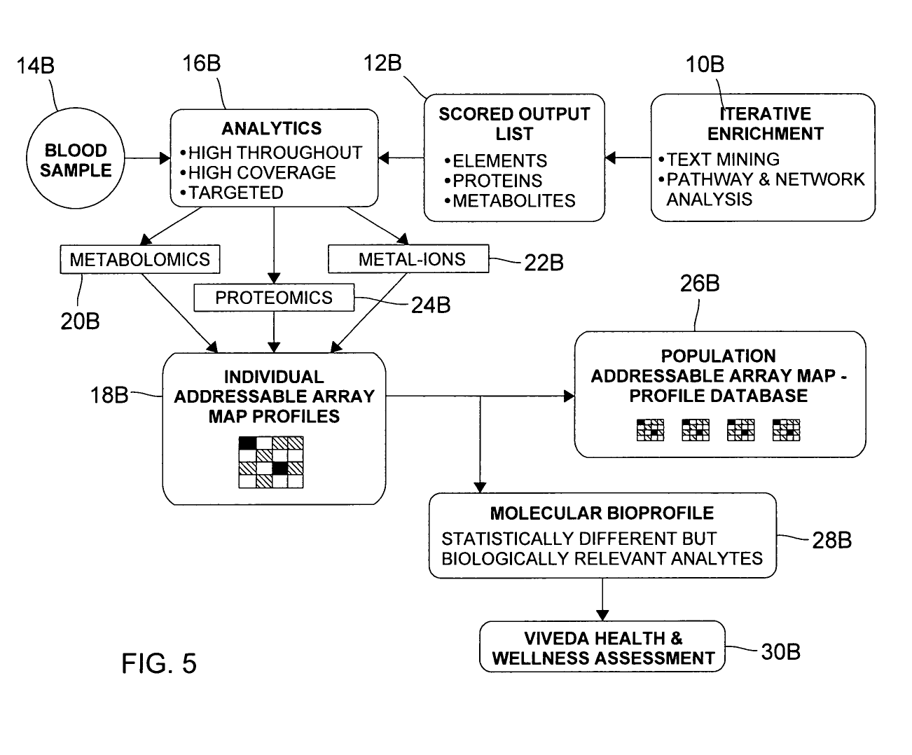

FIG. 5 is a schematic diagram of an exemplary platform for performing methods according to the present disclosure. An exemplary output includes molecular bioprofiling to determine an individual's current state of health and wellness. The exemplary platform includes of a plurality of interconnected modules, including sample collection, sample processing, analytics, mass informatics, bioinformatics, and knowledge assembly modules. A brief synopsis of an exemplary platform's process flow follows.

Initially, knowledge assembly tools 10B are used to create an output list 12B of scored (weighted) molecules and elements (metal ions, for example) to be targeted for profile comparisons that determine individual health and wellness. The list 12B is obtained through the use of knowledge assembly tools 10B, including extensive text mining and/or pathway and network analysis, for example. An exemplary software tool for performing such pathway and network analysis is an Ingenuity Pathways Analysis software tool, commercially available from Ingenuity® Systems (www.ingenuity.com).

Next, an individual biological sample 14B is analyzed using high-throughput analytical instrumentation 16B that provides efficient, targeted coverage and characterization of complex biological samples. Mass informatics and bioinformatics are then used to produce an Addressable Array Map 18B (AAM) for the individual using the datasets 20B, 22B, 24B (e.g., metabolomics 20B, metal ions 22B, proteomics 24B) obtained from the different measurements. The resulting individual AAM 18B is then used in a comparative analysis of individuals against defined populations (i.e., a population AAM 26B). The output is a differential list of molecules with statistically significant differences in concentration between the individual AAM and the population AAMs—known as a molecular bioprofile 28B. In exemplary embodiments, the molecular bioprofile 28B is used to produce a health and wellness assessment 30B, which provides information for individuals and/or clinicians, for example.

An exemplary molecular network making up a molecular bioprofile 28B may include various molecules connected by various relationships, such as direct relationships (e.g., direct regulation), indirect relationships (e.g., indirect regulation), and/or self regulation.

The output of the integrated platform approach using Iterative Enrichment and high-throughput analyses is a molecular bioprofile 28B. The molecular bioprofile provides a biologically related network of bioindicators that determine the biological health and wellness status of the individual compared to a relevant control population. A bioindicator of the present disclosure differs from a conventional biomarker in that a bioindicator has both a defined biological relevance to a health condition and a correlation to other bioindicators as it pertains to the health condition under scrutiny.

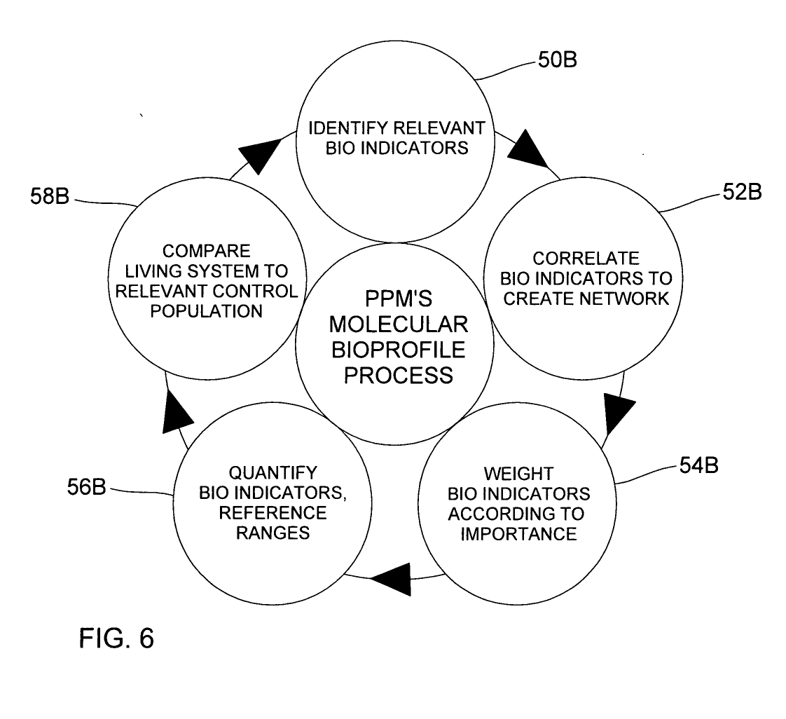

An exemplary molecular bioprofile is constructed as outlined in FIG. 6. A knowledge based text mining search and pathway/network analysis identifies relevant bioindicators 50 (>20, for example) for the health condition being scrutinized. The individual bioindicators are then correlated to create a biologically relevant network 52B and each scored according to the importance of its role in the network 54B. Each bioindicator is also quantified in the biological fluid being measured 56B and compared to normal, healthy ranges for the same analyte 58B. These weighted, correlated and quantified bioindicators form a molecular bioprofile 28B. The importance of the molecular bioprofile 28B is that it identifies and establishes each analyte's biological function in the biologically relevant network associated with each health condition being evaluated—which has significant potential in both predictive and preventive medicine.

An exemplary method of processing information may include identifying a set of relevant bioindicators; correlating the set of relevant bioindicators to create a network; weighting each of the relevant bioindicators in the set according to its importance; analyzing a sample to obtain sample concentrations of each of the relevant bioindicators; and comparing the sample concentrations to respective control concentrations.

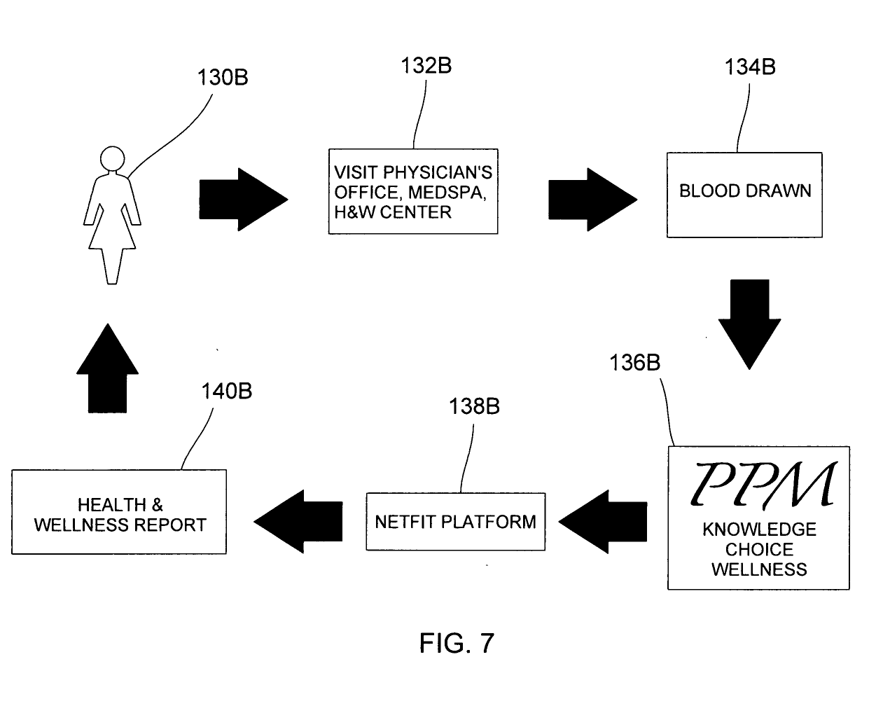

As shown in FIG. 7, an exemplary process begins with an individual 130B utilizing the services of a physician's office, medical spa, health and wellness center 132B to have her blood drawn 134B. The blood sample is measured 136B and the results are analyzed 138B to produce a health and wellness report 140B, which is provided to the individual 130B.

Prior to analysis, a sample may be received by the analysis facility, entered into the laboratory information management system, aliquots may be prepared (in a hood, for example), aliquots may be spun down in a refrigerated centrifuge, for example. Samples and aliquots may be stored in cold conditions, such as at −80° C. Data pertaining to the samples and results of analyses may be stored in a bank of servers, for example.

Exemplary methods include dividing a sample into a plurality of aliquots (some exemplary embodiments utilize 10-12 aliquots, for example). The aliquots are analyzed using instruments, such as a Pegasus 4D GCxGC-TOFMS (a two-dimensional gas chromatograph with a time-of-flight mass spectrometer), a Unique HT TOFMS (a high-throughput liquid chromatograph with a time-of-flight mass spectrometer), a TOF ICPMS (an time-of-flight inductively-coupled plasma mass spectrometer), a Gyrolab Workstation LIF (laser induced fluorescence), and a Roche Cobas MIRA benchtop biochemistry analyzer. It is within the scope of the disclosure to use other analytical instrumentation to analyze samples and to analyze more than one aliquot using a single instrument.

Method of Iterative Enrichment in Information Assembly

This portion of the disclosure relates generally to search approaches for obtaining information, such as information pertaining to diseases or conditions. In particular, this disclosure relates to search approaches that identify both mathematical associations and the contextual relevance of search results.

The present disclosure contemplates that, as methods of analyzing biological fluids were being developed, early methods permitted detection of differences between control samples and diseased samples. However, these early methods did not provide insight into the significance and consequences of the differences between the samples. Such analysis later evolved, and the ability to quantitatively observe the effects of the differences on a molecular basis was developed, but the particular importance of each individual molecule was not known.

The present disclosure contemplates that a contextual problem arises when search and weighting methods are used to generate connectivity between different biological information sets. Specifically, it is not possible to identify the biological context whilst simultaneously ascertaining mathematical associations.

This disclosure includes exemplary methods of processing information that target information in the form of life science descriptors, related molecules, as well as pathways and networks connecting the descriptors and molecules. The resulting information provides both traditional statistical context along with important unconventional biological context.

The present disclosure contemplates that, in contrast, conventional search approaches typically provide only statistical arguments from within information space. Exemplary methods are superior to conventional approaches because they provide biological context to acquired information sets, whilst providing informatically, statistically, and biologically relevant results.

Exemplary methods correlate mathematical associations (M.A.) produced by various search methods with unconventional biological context. In exemplary methods, this may be accomplished by interrogating the mathematical associations against a biological analysis, such as a pathway/network analysis. This allows for the observation of not only the correlations between the mathematical associations, but also the biological context and relevance. The biological context provides insight into the biological nature of the data that is collected throughout the process.

For the Iterative Enrichment (IE) process, the word “enrichment” does not exclusively describe adding information to a collective database. In this process, enrichment can describe both removal from and addition to the database to further enrich the information set.

Exemplary methods can be used in conjunction with any type of search method. The search combs all of a defined information space, such as PubMed journal libraries. The result of the search is mathematical associations between the defined information sets. As an example of mathematical associations, information such as the number of appearances of a molecule in relation to a disease descriptor within PubMed journals can be used in the IE process. In other words, this step utilizes search processes to gain statistical context for an argument. Exemplary methods associate this statistical context and the mathematical associations with biological context. Put another way, exemplary methods seek to answer the question, what do the mathematical associations mean biologically? To this end, the mathematical associations are interrogated against a biological analysis. A pathway and network analysis is an example of such a biological analysis. The rationale for conducting such an analysis is that the mathematical associations can be described in terms of related molecules and biological networks, whilst enriching the information gathered from the initial search.

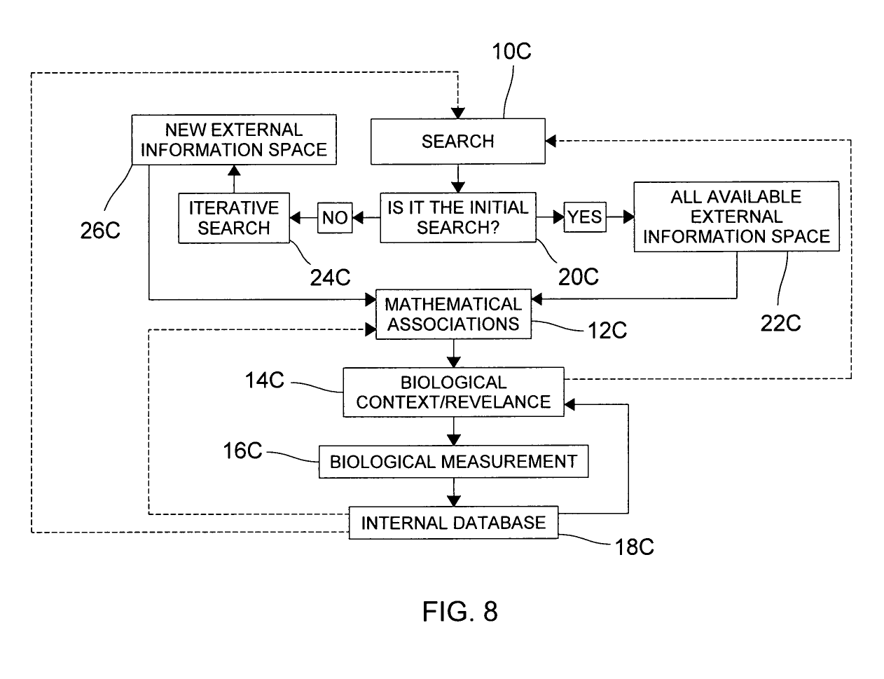

In general, an exemplary method depicted in FIG. 8 begins with a search 10C for mathematical associations 12C. For example, such a search may include text mining and exemplary M.A. include the frequency of molecule appearances in direct relation to disease descriptors within information space.

The biological context and/or relevance 14C of the M.A. are determined using, for example, a pathway/network analysis. The result of this step is that the M.A. are described in terms of relevant pathways, networks, and molecules.

An analytical platform is used to perform a biological measurement 16C. For example, a mass spectrometer may be used to analyze a blood sample. The biological measurement 16C is targeted at molecules identified based on the search list (M.A.) and the pathway/network list (biological context). The resulting information is stored in a database 18C, which may be internal. The information may be stored for later use, such as in additional IE processes and/or further research and development.

In exemplary embodiments, the search process may include steps that vary based on whether the search is an initial search 20C. If so, the search may be performed on all available information space 22C. If the search is not an initial search (an iterative search 24C), the search may be performed on new information space 26C.

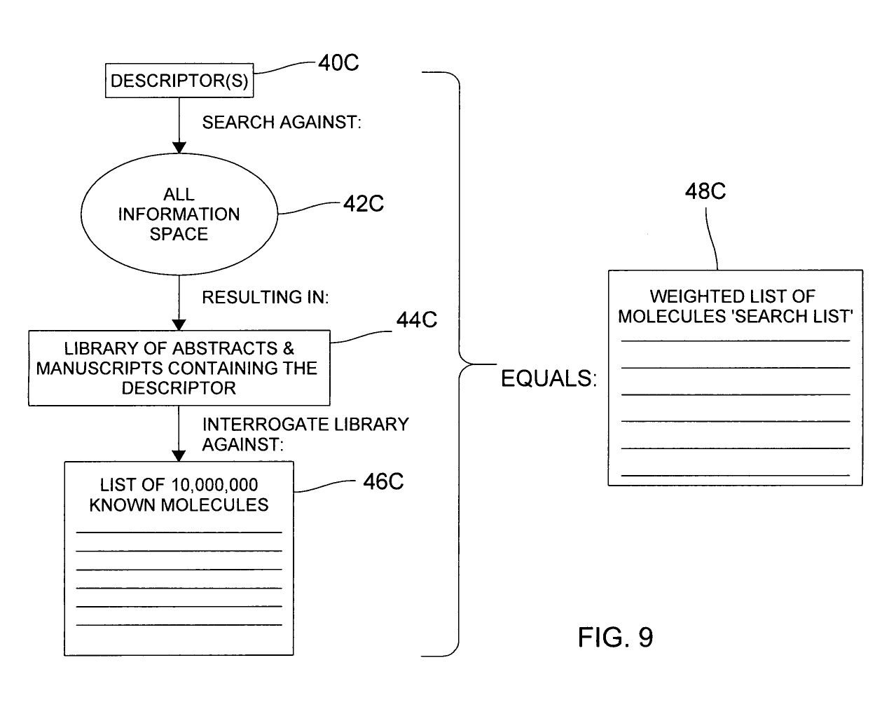

More specifically, an exemplary method including development of a search list is described with reference to FIG. 9. A search list may be a word or list of words that describe the disease or condition that is relevant. For example, a descriptor 40C for the overall condition of stress is “stress”. In exemplary methods, the descriptor “stress” is interrogated against all of available information space 42C. The output of this process is a library 44C of abstracts and manuscripts that contain the descriptor word “stress”. To further the process, the library of abstracts and manuscripts may be interrogated against a list 46 of 10,000,000 known molecules, for example. The two searches connect “stress” to a group of molecules. The result is a library of abstracts and manuscripts that contain the descriptor (stress) and connected molecules.

In an exemplary process, the molecules connected to stress, in the library 44C of abstracts and manuscripts, are scored to provide a weighted list 48C. In an exemplary process, each time a molecule appears in an abstract or manuscript it is scored. For example, a common molecule connected to stress is cortisol. Each time cortisol appears in information space it receives a unit of score. The product is the weighted search list 48C of molecules. In exemplary embodiments, the ranking or weighting factor within the list indicates the frequency and/or type of connection between the molecule and the descriptor.

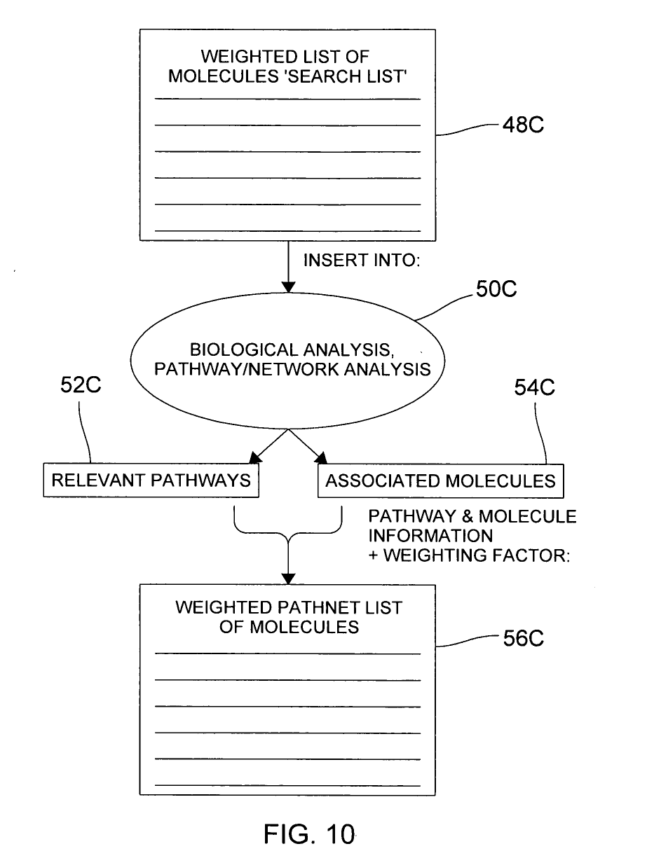

Search results from the above process typically provide no significant arguable biological context. Exemplary methods build on those result by subjecting the most relevant molecules from the search generated list 48C to a biological analysis, such as a pathway/network analysis. As shown in FIG. 10, some or all of the ranked molecules (for example, the top 50%) in the weighted search list 48C may be interrogated against a pathway/network analysis 50C. The molecules are inserted into pathway/network software, generating biological context to the search generated list 48C. The pathway/network software 50C analyzes the relevant pathways 52C and the associated molecules 54C. The result is a pictorial representation of the networks associated with the molecules inserted into the program. Also, a new weighted PathNet list 56C of molecules is generated based on the pathway/network analysis. An exemplary software tool for performing such pathway and network analysis is an Ingenuity Pathways Analysis software tool, commercially available from Ingenuity® Systems (www.ingenuity.com).

As discussed above, the standard flow of information through exemplary iterative enrichment methods begins with a search to discover any mathematical associations. The mathematical associations are then subjected to a biological analysis, such as a pathway/network analysis, to provide the biological context. However, the information processed through the exemplary iterative enrichment processes has no standard operation protocol. Any type of information, from any stage of the IE process, is able to permeate into the overall process at any point and proceed from that point unhindered.

As an example, information may be subjected to the IE process and stored in an internal database 18C. That information could later be withdrawn from the database 18C and subjected to an iterative search of all new available information space 26C. The information would then proceed through the IE process for the second time. This series of events is able to precede an infinite number of times. Each round, the information is enriched through the iterative cycles.

Internal database 18C information can be isolated, searched, and interrogated to determine any mathematical associations within the data. The mathematical associations are then subjected to biological analysis to provide biological context.

An exemplary method of processing information may include searching an information space to identify mathematical associations; performing a biological analysis of the mathematical associations, thereby identifying a list of relevant molecules; measuring concentrations of the relevant molecules in a biological sample; and storing the list of relevant molecules and the concentrations of the relevant molecules in a database.

An exemplary method of processing data may include searching an information space including a plurality of items for at least one descriptor, thereby identifying a set of items containing the descriptor; interrogating the set of items against a list of known molecules, thereby producing a weighted list of molecules; and performing pathway/network analysis on the weighted list of molecules, thereby producing a weighted pathway/network list of molecules.

Method of Scoring and Individual Molecular Bioprofile

This portion of the disclosure relates generally to methods of analyzing data and, more particularly, to methods of scoring molecular bioprofiles. More specifically, this disclosure relates to methods of scoring molecular bioprofiles based on the quantitative measurement of each molecule within a bioprofile relative to its normal concentration range.

The present disclosure contemplates that, historically, health has been defined by the absence or presence of disease. This has led to the current clinical diagnostics sector “single diagnostic marker/disease” model used to determine a person's overall ill-health. More recently, some fledgling attempts have been made to demonstrate the use of biomarker panels (typically 3-10 analytes) as indicators of disease presence and progression. An example is the combined use of creatine kinase-MB, troponin C and C-reactive protein used widely in clinical situations to diagnose myocardial infarction. The individual components are used in an additive manner to increase the specificity and sensitivity of the diagnosis.

In contrast to current clinical diagnostics and biomarker discovery programs, exemplary methods described herein employ a molecular bioprofile of a specific condition in human health, wellness, and disease. An exemplary molecular bioprofile is a network of 20+ biologically correlated and relevant molecules that is indicative of the biological state of a human health condition when it is compared to a control population (or compared to other controls). It is within the scope of this disclosure to utilize a network of many more or potentially fewer biologically correlated and relevant molecules. Exemplary methods generate an individual quantitative score for individual molecular bioprofiles.

In exemplary methods, quantitative scores are scaled on a 0-100 scale, although any scale could be used. Priority scores for each molecule are generated based on processes such as a pathway/network analysis including two graph centrality measurements and information assembly pertaining to known networks. A unified score is calculated based on the results of the processes.

In exemplary methods, knowledge assembly tools are used to create an output list of scored (weighted) molecules and elements (metal ions, for example) to be targeted for profile comparisons that determine individual health and wellness. In exemplary methods, the list is obtained through extensive text mining as well as pathway and network analysis.

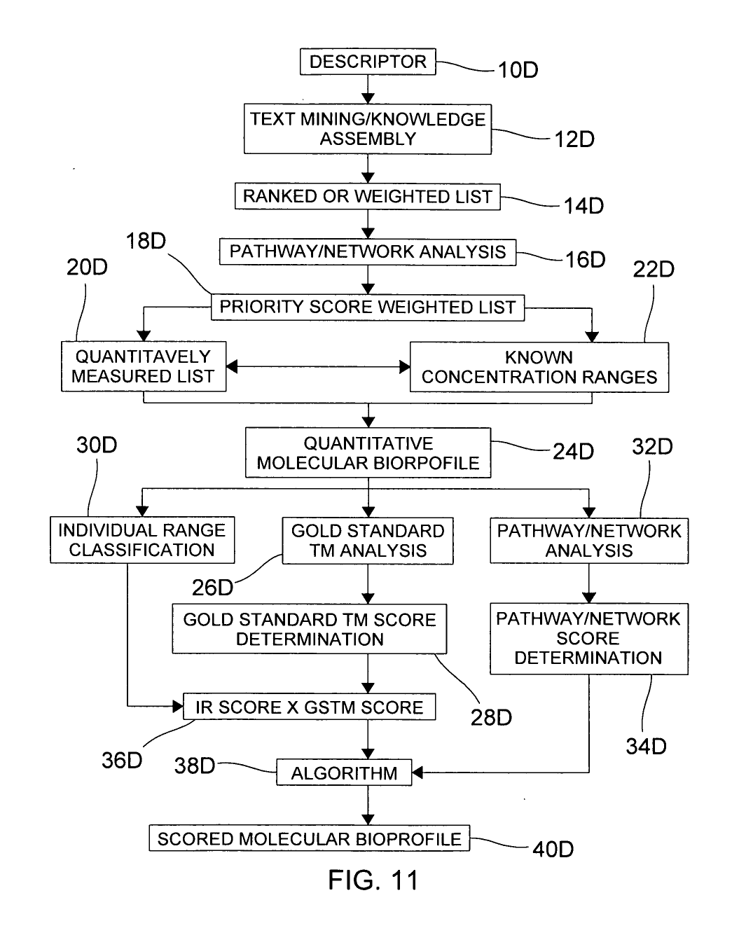

As shown in FIG. 11, an exemplary method begins with selection of at least one descriptor 10D. Text mining and/or other knowledge assembly processes 12D utilize the descriptor 10D to produce a weighted list 14D. The weighted list 14D is subjected to pathway/network analysis 16D, for example, to produce a priority score weighted list 18D.

In exemplary embodiments, an individual biological sample is quantitatively analyzed 20D using high-throughput analytical instrumentation that provides efficient, targeted coverage and characterization of complex biological samples. Mass informatics and bioinformatics are then used to produce an Addressable Array Map (AAM) for the individual using the datasets obtained from the different measurements. The resulting AAM is then used in a comparative analysis of individuals against known concentration ranges 22D for defined populations. The final output is a differential list of molecules with statistically significant differences in concentration between the individual AAM and the population AAMs—known as a molecular bioprofile 24D.

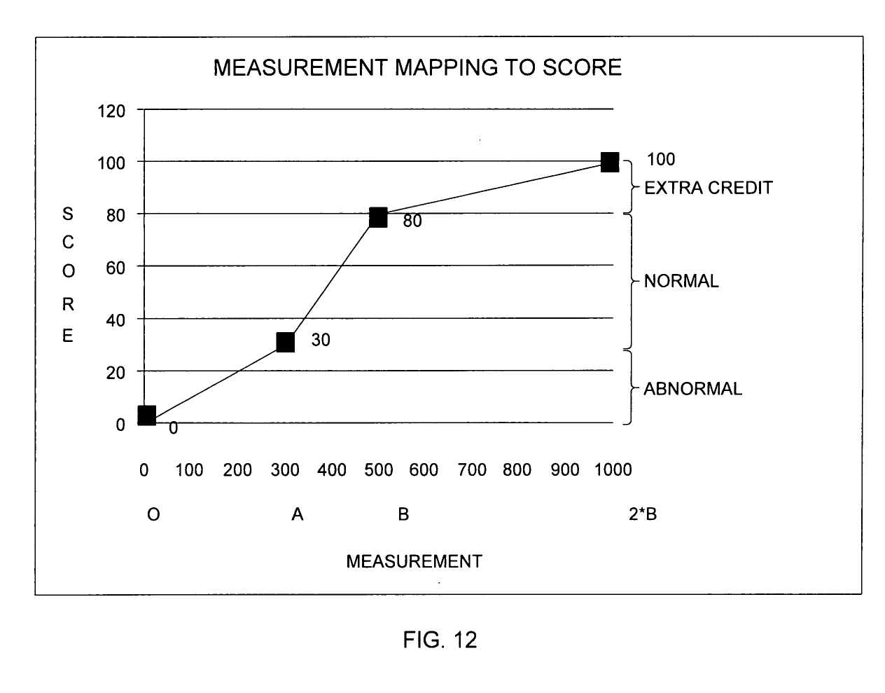

In exemplary embodiments, the molecule's measurements are mapped on a line graph to illustrate the score. An example of such a graph is shown in FIG. 12. In exemplary embodiments, the boundaries of the molecule's normal range are mapped to 30 and 80 on the scoring scale. The abnormal range of the molecule maps to the range 0-30 on the scoring scale and the “better than average” range maps to 80-100. This accommodates the abnormal range lying to the left or the right of the normal range on the graph. In other words, in these exemplary embodiments, the molecular measurements are converted to a 0-100 scale.

In FIG. 12, (a,b) denote the boundaries of the molecule's normal range, 0 and 2*b are the boundaries of the molecule's possible range of measurements. As an example from the graph, (a) begins at 30 and (b) ends at 80. Respectively, the measurements are 300 and 500. Therefore the molecule's possible range of measurements are 0 and 1000 (2*b). The measurements units on the exemplary graph shown in FIG. 2 are arbitrary as the measurement units for each molecule are based on its known reference range. For example, molecule x may have a “normal” reference range of 30-40 units. Therefore, (a) would begin at 30 and (b) would end at 40. Accordingly, the total range of possible measurements for molecule x would be 0 through 80 (2*b).

Scoring molecular bioprofiles and the individual molecules within molecular bioprofiles may be advantageous for many reasons. Scoring allows for easy comparison of individual molecules within a specific molecular bioprofile and also simple comparison of bioprofiles themselves. The overall score also reflects the state of all the molecules together in the form the whole molecular bioprofile. Most importantly the scoring captures the unconventional, and rich system-level understanding of specific health conditions.

In exemplary embodiments, the individual molecular measurements within the bioprofile may have specific characteristics, weighting, and/or justification based mainly on the system-based approach to understanding specific health conditions. For instance, the measurements can span different numerical ranges depending on the molecule being measured. These ranges often differ by orders of magnitude. Attempting to view all the measurements in their original forms at the same time would present a skewed picture and could result in loss of information. Also, while molecules have “normal” ranges within which most measurements are expected to fall, the placement of the normal range within a molecule's overall range can vary between molecules. Higher values might denote abnormality for some molecules whereas the opposite could be true for others. Due to the system-level approach and the inherent characteristics of a molecular bioprofile, all molecules do not make equal contributions to the presence or absence of a disease. Thus, it could be inaccurate and misleading to weight the contributions from all molecules equally.

In the exemplary method shown in FIG. 11, individual ranges for the measured molecules are determined 30. For example, all scores of the individual molecules may be scaled to fall on a numerical range of 0 through 100. This enhances comparability between scores. More specifically, in exemplary embodiments, scores between 30 and 80 denote normal measurements. Any score greater than 80 is a “better than average” reading. Scores less than 30 indicate abnormal readings. This allows scores to be interpreted in the same fashion, irrespective of origin.

Given the candidate set of molecules associated with a health condition, it may be helpful to identify those molecules within the set that are most indicative of the health condition. This may be accomplished via several processes. The first is known as Gold Standard Text-Mining (GSTM). Historically, gold standard molecules of a specific condition within health and wellness are classified as critical to that specific condition. For example, gold standard molecules for Type II Diabetes are insulin, glucose and hemoglobin A1C. The importance of these GSTM molecules, with respect to the overall bioprofile for a given condition, is reflected in the GSTM priority contribution.

In exemplary methods, the molecular bioprofile 24D is subjected to gold standard text mining analysis 26D, which determines the GSTM score 28D. In particular, the molecules identified in the molecular bioprofile 24D as having statistically abnormal concentrations may be analyzed using Gold Standard Text Mining. This text mining may be performed on the same corpus that was mined to generate the list of scored molecule and elements, for example. In exemplary embodiments, this text mining may utilize as descriptors terms such as the molecules identified in the molecular bioprofile 24D as having statistically abnormal concentrations, disease specific terms, and/or terms such as and related to “gold standard.” Thus, in exemplary embodiments, the GSTM score 28D for each molecule having an abnormal concentration is indicative of the relative importance of that molecule to a particular disease or condition.

Secondly, the priorities are also determined from publicly available networks of molecular interactions by performing pathway/network analysis 32D. Due to the fact that the generated networks can be treated like graphs, two measures of graph centrality are used to ascertain the relative contributions of the molecules in exemplary embodiments. The degree of centrality is determined for each molecule. Degree centrality is defined as the number of links incident upon a node. In other words, for a specific molecule, the number of links to other molecules within the network is determined. Secondly, each molecule is subjected to a betweenness analysis. Betweenness is a centrality measure of a vertex within a graph. Vertices that occur on many shortest paths between other vertices have higher betweenness than those that do not. Using these or other methods, a pathway/network score is determined 34D. An exemplary software tool for performing such pathway and network analysis is an Ingenuity Pathways Analysis software tool, commercially available from Ingenuity® Systems (www.ingenuity.com).

In exemplary embodiments, the product 36D of the individual range score 30D and the GSTM score 28D is combined with the pathway/network score 34D in an algorithm 38D. The result is a scored molecular bioprofile 40D.

Each molecule has a priority or importance with respect to a given health condition. In the exemplary embodiment, these are expressed as fractions that sum to 1 for all molecules associated with a condition. This priority allows for weighting of contributions from different molecules to the overall profile and also captures the system's level understanding of the condition.

In exemplary embodiments, the unified score for all the molecules is calculated by integrating the molecular priorities and the individual scores of the molecules. The priority serves as a weighting factor and performs the important function of penalizing a molecule falling in the abnormal range in accordance with its perceived importance to the disease. This also increases the unified score of a molecule falling in the normal or “better than average” ranges significantly if the molecule is important.

The actual computation of the score is:

$${S}^{D} = {\sum }_{i = 1}^{{n}^{D}} {P}_{i}^{D} * {S}_{i}^{D}$$

S^{D}—Unified score of individual for disease D
n^{D}—Number of molecules measured for disease D
Pᵢ^{D}—Priority/Importance of molecule i for disease D such that

$${\sum }_{i = 1}^{{n}^{D}} {P}_{i}^{D} = 1$$

Sᵢ^{D}—Scaled score of molecule i for disease D. This is computed by a linear mapping of the molecule's measurement to the appropriate scaled score range.

In exemplary embodiments, the unified score also falls on a scale of 0 through 100. If all of the molecular measurements fall within the normal range, the individual scaled scores and the unified score will also be in the normal range (30-80). If the unified score is greater than 80, one or more individual scaled scores are greater than 80 (i.e., fall in the “better than average” range). If the unified score is less than 30, one or more individual scaled scores are less than 30 (i.e., fall in the abnormal range). In exemplary embodiments, a plurality of unified scores may be generated, each of which relates to a particular disease or health condition.

As an example, a health condition with four relevant molecules and their respective scores will appear as follows

| Priority | Scaled Score | Contribution to Total Score | Normal? |
|---|---|---|---|
| 0.45 | 20 | 9 | No |
| 0.3 | 40 | 12 | Yes |
| 0.15 | 60 | 9 | Yes |
| 0.1 | 30 | 3 | No |

$$\begin{matrix}Unified score = 0.45 * 20 + 0.3 * 40 + 0.15 * 60 + 0.1 * 30 \\ = 9 + 12 + 9 + 3 \\ = 33\end{matrix}$$

From the above example, it is apparent that a low priority molecule that is abnormal has a much lower contribution to the score than a high priority molecule which is abnormal.

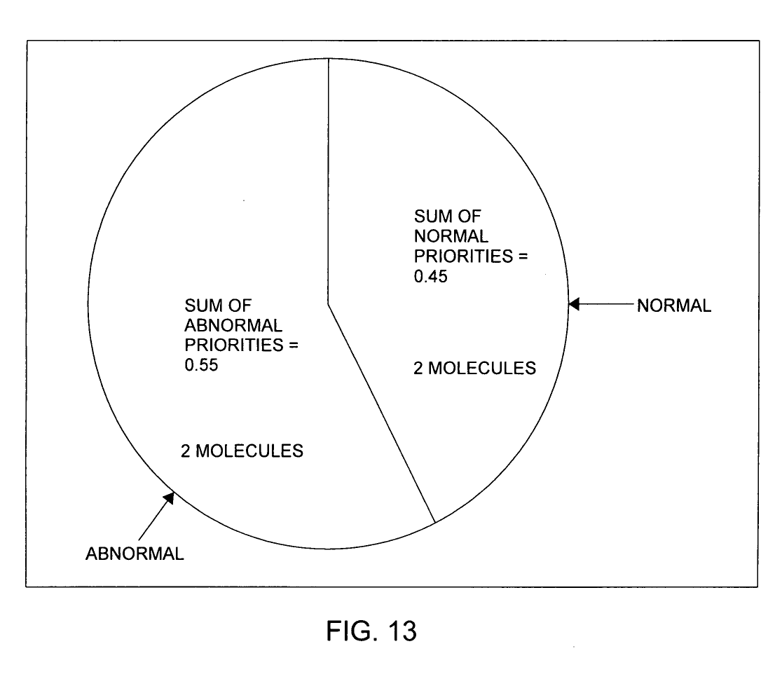

Interpretation of the unified score is assisted by a pie chart listing both the accumulated priority and the number of molecules falling into normal and abnormal ranges. FIG. 13 is an exemplary pie chart. This allows an estimate of whether the molecules falling into each range had low or high priorities individually. Fewer molecules in a range that has a high priority total imply high individual priorities for one or more of the molecules.

An exemplary method of processing information may include generating a priority score weighted list of molecules; analyzing a biological sample to measure a respective sample concentration of each molecule on the priority score weighted list; scoring the sample concentrations, thereby generating respective molecule scores; and calculating a unified score.

An exemplary method of processing information includes generating a weighted list of molecules; performing pathway/network analysis on the weighted list of molecules to produce a priority score weighted list of molecules; analyzing a biological sample to obtain sample concentrations of the molecules on the priority score weighted list; comparing each of the sample concentrations to a respective control value to identify statistically significant differences, thereby producing a molecular bioprofile; calculating an individual range score for each molecule in the molecular bioprofile by scaling the respective sample concentration; calculating a text mining score for each molecule in the molecular bioprofile by text mining a corpus; calculating a pathway/network score for each molecule in the molecular bioprofile by performing pathway/network analysis on the molecules in the molecular bioprofile; and combining the individual range score, the text mining score, and the pathway/network score to yield a scored molecular bioprofile.

Preparation of Medical Data Reports

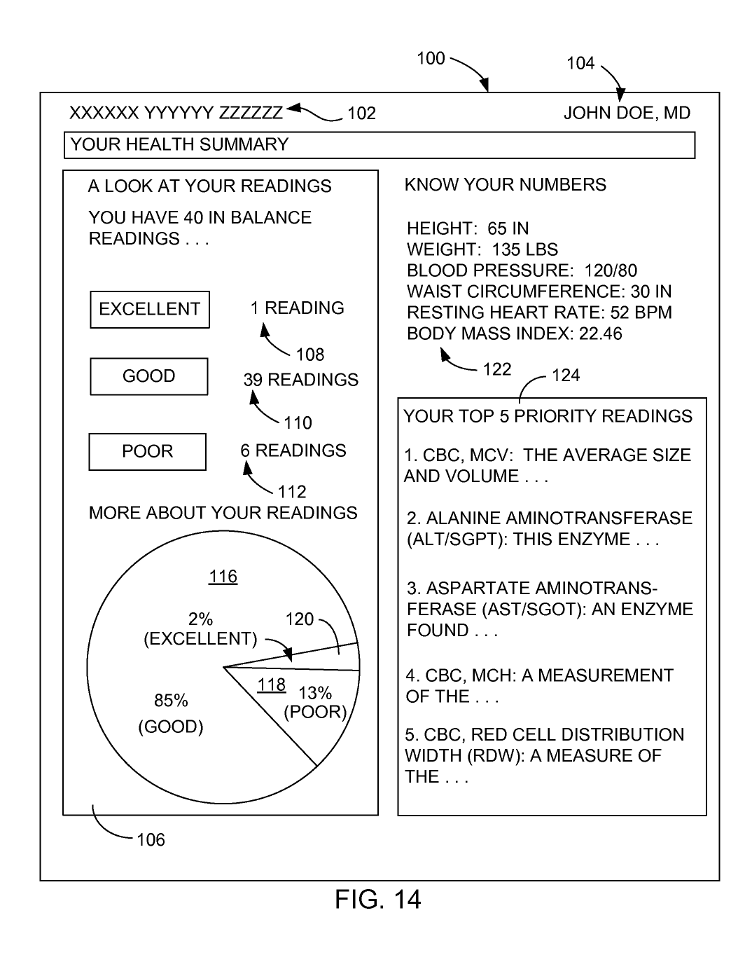

FIG. 14 illustrates an example health summary page 100 according to the present disclosure. Health summary page 100 may include patient identifying information 102, such as a patient name, a patient identifier (e.g., patient number, social security number, etc.), a medical record identifier (e.g., an number or an alpha-numeric sequence associated with the patient's medical record), sample identifier (e.g., a number and/or an alpha-numeric sequence associated with one or more samples associated with the results included in the report), and/or a results access key (e.g., a unique alpha-numeric identifier that allows a patient online access to a specific report). Some example health summary pages 100 may include other identifying information, such as the name of the ordering physician 104, the name of a medical facility, or the like.

Some example health summary pages 100 may include a numerical summary section 106, which may include numerical summary data, such as numbers 108, 110, 112 of excellent readings, good readings, and poor readings, respectively. Some example numerical summary sections 106 may include text explaining the numerical summary section 106 to the reader, such as the following:
You have 40 in balance readings (good or excellent results) and 6 out of balance readings (poor results). Individual results for each reading are in the readings section of this report.

Some example numerical summary sections 106 may include a graphical depiction of the numerical summary data, such as in the form of a pie chart 114. An example pie chart 114 may include portions 116, 118, 120 corresponding to the numerical summary data, such as the numbers 108, 110, 112 of excellent readings, good readings, and poor readings. In some example embodiments, a legend may list the correlation between colors, symbols, and the like used in the graphical depiction with the categorization of the readings. An example numerical summary section 106 may include text explaining the chart, such as the following:
This chart indicates the percentage of your readings that were poor, good, and excellent.

Some example health summary pages 100 may include one or more sections 122 providing other information, such as the patient's height, weight, blood pressure, waist circumference, resting heart rate, and/or body mass index. In some example embodiments, such sections 122 may include text, such as the following:
Keep track of these important numbers recorded at the time of your lab tests.
Your height and weight measurements have been used to determine your body mass index (BMI). Body mass index compares your height and weight to help determine whether you are under- or over-weight. A BMI of less than 18.5 indicates that you are under-weight, 18.5-24.9 is normal, and anything over 25 indicates that you are over-weight.

Some example health summary pages 100 may include a priority readings section 124, which may include information pertaining to readings that may be particularly important. For example, a priority readings section 124 may include readings that are outside of normal limits. Some example priority readings sections 124 may include explanatory text, such as the following:
This report includes 46 lab test readings. The readings below represent your most critical results and should be given priority attention by you and your physician.

In some example embodiments, a priority readings section 124 may provide identifying information pertaining an individual priority reading (e.g., the name of the analyte or test, such as “CBC, MCV”) and/or information pertaining to the reading. In some example embodiments, the priority readings section 124 may include a numbered list of priority readings. For example, a priority readings section 124 may include the following text:
1. CBC, MCV: The average size and volume of your red blood cells, A diet rich in iron and vitamin B12-promotes a healthy MCV. Beef and seafood are excellent B12 sources.
2. Alanine Aminotransferase (ALT/SGPT): This enzyme provides information on how your liver is functioning, an important health indicator.
3. Aspartate Aminotransferase (AST/SGOT): An enzyme found throughout your body, aspartate aminotransferase is a good indicator of your liver function and health.
4. CBC, MCH: A measurement of the amount of hemoglobin in your red blood cells. Foods rich in folic acid, such as spinach and sunflower seeds, help maintain healthy MCH amounts.
5. CBC, Red cell distribution width (RDW): A measure of the variation in width among your body's red blood cells. Iron and vitamin-rich foods, such as meats and leafy vegetables, aid development of healthy red blood cells.

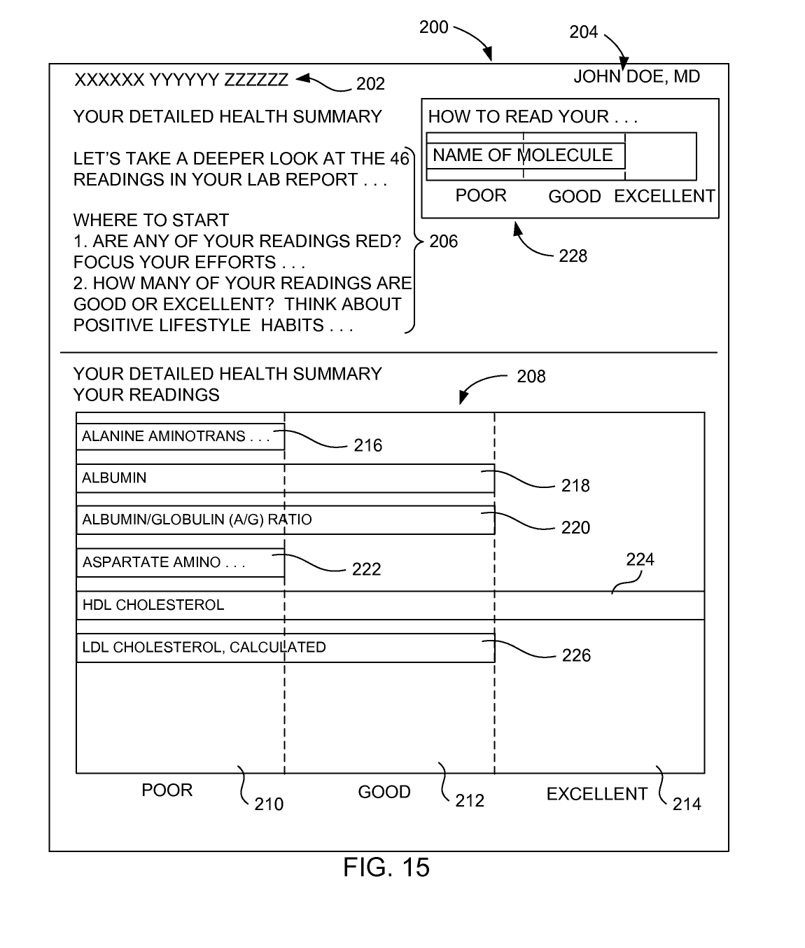

FIG. 15 illustrates an example detailed health summary page 200 according to the present disclosure. Detailed health summary page 200 may include patient identifying information 202, such as such as a patient name, a patient identifier, a medical record identifier, sample identifier, and/or a results access key. Some example detailed health summary pages 200 may include other identifying information, such as the name of the ordering physician 204, the name of a medical facility, or the like.

Some example detailed health summary pages 200 may include instructions 206 to the patient pertaining to, for example, interpretation of the report. Such instructions 206 may include, for example:
Let's take a deeper look at the 46 readings in your lab report. Your readings are categorized as poor, good, or excellent as compared to an acceptable, healthy range.
Where to start
1. Are any of your readings red? Focus your efforts on improving these poor readings first.
2. How many of your readings are good or excellent? Think about positive lifestyle habits that help you maintain these readings.

Some example detailed health summary pages 200 may include graphical representations of the categorizations associated with individual readings. For example, a bar graph section 208 may include vertical columns 210, 212, 214 associated with poor, good, and excellent categorizations, respectively. Individual readings may be represented as horizontal bars 216, 218, 220, 222, 224, 226, which may extend across columns 210, 212, 214 to reflect the categorization of the individual readings. For example, an example reading for Alanine Aminotransferase (ALT/SGPT) may be categorized as poor, and the respective horizontal bar 216 may extend across the poor column 210. An example reading for Albumin may be categorized as good, and the respective horizontal bar 218 may extend across the poor column 210 and the good column 212. An example reading for HDL Cholesterol may be categorized as excellent, and the respective horizontal bar 224 may extend across the poor column 210, the good column 212, and the excellent column 214. Instructions 228 for reading the graphical representations may be provided on some example detailed health summary pages 200.

Some example detailed health summary pages 200 (and/or any other pages of an example report) may employ one or more visually distinguishable characteristics in connection with readings and/or characterizations (e.g., color-coding, shading, graphical symbols and/or the like). For example, portions of various bar graphs and/or pie charts associated with readings categorized as poor may be shaded with a first color (e.g., red), portions associated with readings categorized as good may be shaded with a second color (e.g., green), and/or portions associated with readings categorized as excellent may be shaded with a third color (e.g., blue).

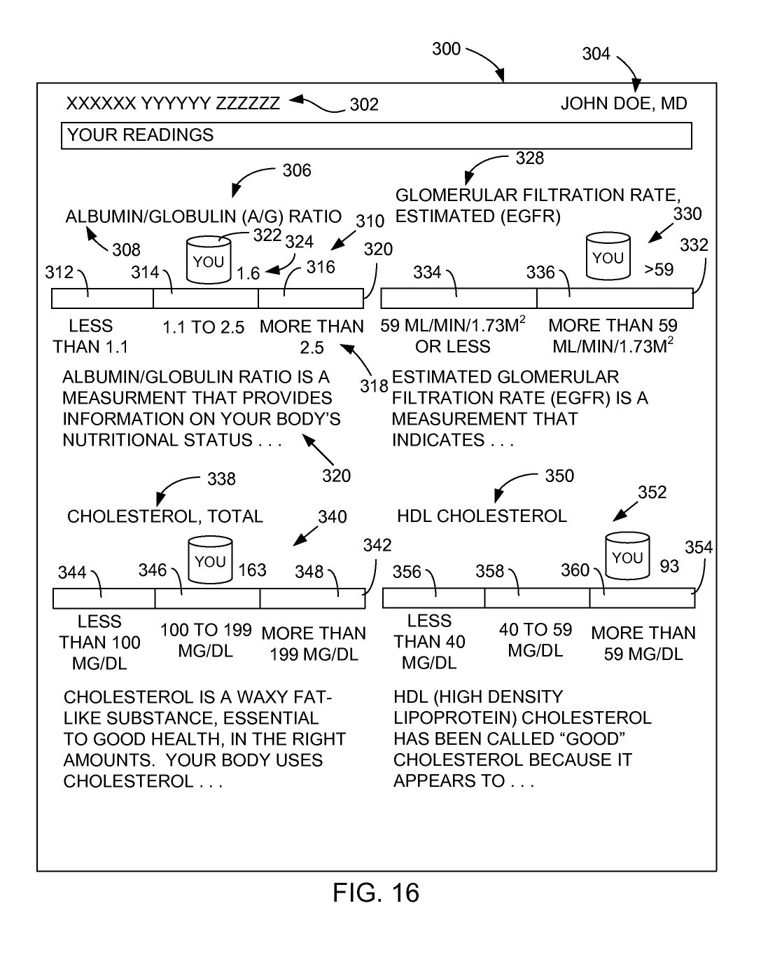

FIG. 16 illustrates an example readings page 300 according to the present disclosure. Readings page 300 may include patient identifying information 302, such as such as a patient name, a patient identifier, a medical record identifier, sample identifier, and/or a results access key. Some example detailed health summary pages 300 may include other identifying information, such as the name of the ordering physician 304, the name of a medical facility, or the like.

Some example readings pages 300 may include information associated with an individual reading. For example, information associated with albumin/globulin (A/G) ratio 306 may include a reading name 308, a graphical representation of the reading 310, and/or text 320, which may provide information about the reading. An example graphical representation 310 may include a scale 326, which may be divided into two or more segments 312, 314, 316, which may correspond with respective categorizations (e.g., poor, good, excellent). Labels 318 may be associated with one or more of the segments 312, 314, 316 and may provide values associated with each segment. An indicator 322 may be placed on and/or adjacent the scale 326 to illustrate the approximate value of the reading and/or in which segment the reading falls. In some example embodiments, a numerical indication of the reading 324 may be provided.

Some example embodiments may include text 320, such as the following information pertaining to the albumin/globulin (A/G) ratio:
Albumin/Globulin Ratio is a measurement that provides information on your body's nutritional status. It's also used to evaluate kidney and liver health. When albumin levels drop, your body increases globulin levels in an effort to maintain a normal total protein level. An abnormal albumin/globulin ratio indicates an imbalance of the types of proteins present in your blood.

Example graphical representations may be configured to reflect categorizations associated with individual readings. For example, in the example information associated with albumin/globulin (A/G) ratio 306, the graphical representation of the reading 310 may include a scale 326 divided into three segments 312, 314, 316, in which two of the segments 312, 316 are associated with a poor categorization and segment 314 is associated with a good categorization. In the example information associated with glomerular filtration rate, estimated (EGFR) 328, the graphical representation of the reading 330 may include a scale 332 divided into two segments 334, 336, in which segment 334 is associated with a poor categorization and segment 336 is associated with a good categorization. In the example information associated with cholesterol, total 338, the graphical representation of the reading 340 may include a scale 342 divided into three segments 344, 346, 348, in which segment 344 is associated with an excellent categorization, segment 346 is associated with a good categorization, and segment 348 is associated with a poor categorization. In the example information associate with HDL cholesterol 350, the graphical representation of the reading 352 may include a scale 354 divided into three segments 356, 358, 360, in which segment 356 is associated with a poor categorization, segment 358 is associated with a good categorization, and segment 360 is associated with an excellent categorization.

Some example reports may include a plurality of readings pages 300, each of which may include information associated with a plurality of individual readings. Example individual readings and example text associated with the individual readings follow:
alanine aminotransferase (alt/sgpt)
good: 0 to 40 IUnits/L/poor: More than 40 IUnits/L
Alanine Aminotransferase (ALT) is an enzyme that is normally present at low levels in your body. Increased amounts of ALT in your body can be a result of liver or heart damage. Some medications also elevate ALT levels. This enzyme is an important indicator of liver health.
albumin
poor: Less than 3.5 g/dL/good: 3.5 to 5.5 g/dL/poor: More than 5.5 g/d L
Albumin is a protein produced in your liver that acts as a transporter. It transports essential fatty acids to your muscles and moves hormones, drugs, and other small molecules through your blood. The most abundant protein in your blood, it also keeps the fluid in your blood from leaking into tissue. Decreased levels can indicate problems with your kidney or liver, infection, malnutrition or the effects of a low protein diet. Eating high protein foods can help increase the amount in your body.
albumin/globulin (a/g) ratio
poor: Less than 1.1/good: 1.1 to 2.5/poor: More than 2.5
Albumin/Globulin Ratio is a measurement that provides information on your body's nutritional status. It's also used to evaluate kidney and liver health. When albumin levels drop, your body increases globulin levels in an effort to maintain a normal total protein level. An abnormal albumin/globulin ratio indicates an imbalance of the types of proteins present in your blood.
alkaline phosphatase
poor: Less than 25 IUnits/L/good: 25 to 150 IUnits/L/poor: More than 150 IUnits/L
Alkaline Phosphatase (ALP) is an enzyme produced by your liver, but also made by your bones, intestines, and kidneys. It is released by your body into the blood during injury and normal activities such as bone growth and pregnancy. Your physician uses it to assess your liver health. Bone or liver disease, heart problems, infection, alcohol consumption, and certain medications can raise the amounts in your body.
amylase, serum
poor: Less than 31 Units/L/good: 31 to 124 Units/L/poor: More than 124 Units/L
Amylase is an enzyme produced mainly by your pancreas and the saliva glands in your mouth. This enzyme is responsible for breaking down the starch you eat into sugar. Common conditions that can cause an increase in amylase levels include inflammation of your gall bladder, ulcers, and inflammation or damage to your pancreas. Decreases in amylase levels occur with hepatitis, alcoholism, and kidney disease.
anion gap
poor: Less than 7 mmol/L/good: 7 to 16 mmol/L/poor: More than 16 mmol/L
Anion Gap is a measurement of the positively and negatively charged ions in your blood. Sodium and potassium, known as cations, are positively charged. Chloride and bicarbonate, called anions, are negatively charged. To maintain optimal health, your body requires a balance between cations and anions.
aspartate aminotransferase (ast/sgot)
good: 0 to 40 IUnits/L/poor: More than 40 IUnits/L
Aspartate Aminotransferase (AST) is an enzyme that naturally circulates in your body. It can be found in your red blood cells, liver, heart, muscle tissue, pancreas, and kidneys. Normally present in a low amount, damage or disease in the heart and liver can increase AST levels. Medications and excessive alcohol use can also increase the amount of AST in your body.
bilirubin, total
poor: Less than 0.1 mg/dL/good: 0.1 to 1.2 mg/dL/poor: More than 1.2 mg/dL
Bilirubin is a compound found in bile that is produced when the liver breaks down old red blood cells. When the body overproduces bilirubin or the liver cannot process it, jaundice occurs. Your physician uses bilirubin concentration to determine whether your liver is functioning properly.
blood urea nitrogen
poor: Less than 5 mg/dL/good: 5 to 26 mg/dL/poor: More than 26 mg/dL
Blood Urea Nitrogen (BUN) is a test routinely used to evaluate kidney function. Urea nitrogen is a waste product of protein metabolism found in your blood. Urea is formed by the liver and carried through your bloodstream to the kidneys for excretion. Increased levels in a BUN test may also indicate dehydration, stress, excessive protein intake or even heart problems. Decreased levels are rare, but could be caused by pregnancy, anabolic steroid use, low protein diet, malnutrition or liver disease.
bun/creatinine ratio
poor: Less than 8/good: 8 to 27/poor: More than 27
BUN/Creatinine Ratio is a comparison between your BUN (Blood Urea Nitrogen) level and creatinine level. Physicians often use this ratio to check for kidney damage, dehydration or problems with kidney function.
calcium
poor: Less than 8.5 mg/dL/good: 8.5 to 10.6 mg/dL/poor: More than 10.6 mg/dL
Calcium (Ca2+) is the most abundant mineral in your body. More than 99% of the calcium in your body is stored in the bones and teeth. Calcium is crucial to building and maintaining strong bones, but it also serves other important functions. It allows your blood to clot, muscles to contract, nerves to send messages, and your brain to process information. Studies have also shown that calcium contributes to healthy blood pressure. Your body gets the calcium it needs in two ways. The most important method is through consumption of calcium-rich foods including dairy products and dark, leafy greens. If your diet is calcium deficient your body will get what it needs from your bones, which can weaken bone structure.
carbon dioxide, total
poor: Less than 20 mmol/L/good: 20 to 32 mmol/L/poor: More than 32 mmol/L
Total Carbon Dioxide is a chemical compound that acts as a buffer to maintain normal levels of acidity (pH) in blood and other fluids in your body. Your body's acidity can be affected by foods or medication and by kidney and lung function. High levels can indicate chronic obstructive pulmonary disease (COPD), fluid in the lungs and heart disease. Hyperventilation, liver or kidney disease, hyperthyroidism, or uncontrolled diabetes can result in low levels of bicarbonate, a component of carbon dioxide.
cbc, hemoglobin
poor: Less than 11.5 g/dL/good: 11.5 to 15.0 g/dL/poor: More than 15.0 g/dL
Hemoglobin is one of the most abundant proteins in your red blood cells. Hemoglobin is used by your red blood cells to transport oxygen from your lungs to the tissues in your body. In addition to oxygen, it's also used to transport other gases and nutrients. Vitamins and minerals such as vitamin C and iron are essential to maintaining the right amount of hemoglobin in your body.
cbc, hematocrit
poor: Less than 34.0%/good: 34.0 to 44.0%/poor: More than 44.0
Hematocrit is a measurement of your total blood volume that is occupied by red blood cells. Elevated hematocrit can indicate dehydration, while low amounts can be a sign of vitamin and mineral deficiencies or anemia. Drinking plenty of water and eating a balanced diet are important to maintaining a healthy hematocrit.
cbc, mchc
poor: Less than 32.0 g/dL/good: 32.0 to 36.0 g/dL/poor: More than 36.0 g/dL
Mean Corpuscular Hemoglobin Concentration (MCHC) measures the amount of hemoglobin in a specific volume of packed red blood cells. Packed red blood cells are RBCs that have been completely separated from your whole blood. Hemoglobin is a protein in your red blood cells that distributes oxygen from your lungs throughout your body. Maintaining healthy levels of hemoglobin is important for sustaining many critical bodily functions. Fruits and vegetables containing vitamin C are a great way to support healthy hemoglobin amounts in your body.
cbc, mch
poor: Less than 27.0 pg/good: 27.0 to 34.0 pg/poor: More than 34.0 pg
Mean Corpuscular Hemoglobin (MCH) is a measurement to determine the amount of hemoglobin in your red blood cells. Hemoglobin is a protein in your blood that carries oxygen from your lungs to the rest of your body. Foods rich in folic acid, such as spinach and sunflower seeds, are essential to maintaining healthy mean corpuscular hemoglobin.
cbc, platelets
poor: Less than 140/good: 140 to 450/poor: More than 450
Platelets, created in your bone marrow, help your blood to clot by plugging damaged areas within your blood vessels. Insufficient platelets in your blood can cause abnormal bleeding or bruising. A diet high in omega-3 fatty acids, fruits, and vegetables helps your body produce adequate numbers of platelets.
cbc, mcv
poor: Less than 80 fL/good: 80 to 98 fL/poor: More than 98 fL
Mean Corpuscular Volume (MCV) is the average size and volume of your red blood cells. Mean corpuscular volume is determined by comparing the volume of your red blood cells to the total volume of your whole blood. An iron and vitamin B12-rich diet is very important to a healthy MCV. Beef and seafood are excellent sources of vitamin B12. Beef, particularly liver, and dark leafy vegetables are also good sources of iron.
cbc, red cell distribution width (rdw)
poor: Less than 11.7%/good: 11.7 to 15.0%/poor: More than 15.0%
Red Blood Cell (RBC) Distribution Width measures size variation of the red blood cells in your body. It is normal to have some small differences in the size of your red blood cells, but conditions like sickle cell anemia cause large variations in red blood cell size. Iron and vitamin-rich foods, such as meats and leafy vegetables, help your body develop healthy red blood cells.
cbc, red blood cells (rbc)
poor: Less than 3.80×10E6/μL/good: 3.80 to 5.10×10E6/μL/poor: More than 5.10×10E6/μL
Red blood cells (RBC) are vital to your health because they are responsible for transporting oxygen from your lungs throughout your body. A diet low in iron, copper and B-vitamins can lead to a decreased number of red blood cells.
cbc/diff, band neutrophils, absolute count
good: 0.0 to 0.1×10E3/μL/poor: More than 0.1×10E3/μL
Band neutrophils are the fifth cell type formed during the development of neutrophils. Neutrophils are cells in your body that fight bacterial infection. A small number of band cells are present in the blood of healthy individuals. The band neutrophil absolute count is the actual number of band neutrophils present in your blood. An increase in the number of band neutrophils in the blood occurs during pregnancy and often indicates a response to inflammation or infection.
cbc, white blood cell (wbc)
poor: Less than 4.0×10E3/μL/good: 4.0 to 10.5×10E3/μL/poor: More than 10.5×10E3/μL
White Blood Cells (WBC) serve important roles as defense mechanisms for your body. Along with red blood cells, white blood cells are made in your bone marrow. Critical to your immune system, your body uses them to battle bacterial and viral infections. Foods like beef, fruits and vegetables are rich in zinc, which helps support healthy white blood cells. Food seasonings such as sesame and mustard seeds are also a great way to add zinc to your diet and improve your white blood cell count.
cbc/diff, basophils, absolute count
good: 0.0 to 0.2×10E3/μL/poor: More than 0.2×10E3/μL
Basophils are a type of white blood cell produced by your immune system in response to inflammation caused by allergic reactions. They migrate to the site of the allergic reaction and release molecules to relieve the inflammation. The basophil absolute count is the actual number of basophils present in your blood.
cbc/diff, band neutrophils, percent
good: 0 to 3%/poor: More than 3%
Band neutrophils are the fifth cell type formed during the development of neutrophils. Neutrophils are cells in your body that fight bacterial infection. A small number of band neutrophils are present in the blood of healthy individuals. The band neutrophil percent is the percentage of white blood cells that are band neutrophils. An increase in the number of band neutrophils in the blood occurs during pregnancy and often indicates a response to inflammation or infection.
cbc/diff, blast, absolute count
good: 0%/poor: More than 0%
Blasts are young blood cells. Blasts are the first cell type created during the development of several different types of cells in your body. The blast absolute count is the actual number of blast cells present in your blood. Finding a blast cell in a healthy person's blood sample is rare. Your doctor can explain the significance of these cells in your blood.
cbc/diff, basophils, percent
good: 0 to 3%/poor: More than 3%
Basophils are a type of white blood cell produced by your immune system in response to inflammation caused by allergic reactions. They migrate to the site of the allergic reaction and release molecules to relieve the inflammation. The basophil percent is the percentage of white blood cells that are basophils.
cbc/diff, eosinophils, absolute count
good: 0.0 to 0.4×10E3/μL/poor: More than 0.4×10E3/μL
Eosinophils are a type of white blood cell used by your immune system to fight viral and parasitic infections. The eosinophil absolute count is the actual number of eosinophils present in your blood. High levels of eosinophils in your body can indicate a parasitic infection.
cbc/diff, blast, percent
good: 0%/poor: More than 0%
Blasts are young blood cells. Blasts are the first cells type created during the development of several different types of cells in your body. The blast percent is the percentage of white blood cells that are blast cells. Finding a blast cell in a healthy person's blood sample is rare. Your doctor can explain the significance of these cells in your blood.
cbc/diff, lymphocytes, absolute count
poor: Less than 0.7×10E3/μL/good: 0.7 to 4.5×10E3/μL/poor: More than 4.5×10E3/μL
Lymphocytes are a type of white blood cell used by your immune system to protect against viral infections. The lymphocyte absolute count is the actual number of lymphocytes present in your blood. High amounts of lymphocytes can indicate viral infection. Zinc, along with vitamins B and C are important to supporting a healthy immune system. Seafood, fruits and vegetables are good sources of zinc, vitamin B and vitamin C.
cbc/diff, eosinophils, percent
good: 0 to 7%/poor: More than 7%
Eosinophils are a type of white blood cell used by your immune system to fight viral and parasitic infections. The eosinophil percent is the percentage of white blood cells that are eosinophils. High levels of eosinophils in your body can indicate a parasitic infection.
cbc/diff, metamyelocyte, absolute count
good: 0.0×10E3/μL/poor: More than 0.0×10E3/μL
Metamyelocytes are the forth cell type formed during the development of neutrophils, basophils, and eosinophils, which are the cells in your body that fight bacterial infection. The metamyelocyte absolute count is the actual number of metamyelocytes present in your blood. Metamyelocytes are typically found in your bone marrow and usually are not present in your blood. Your doctor can explain the significance of these cells in your blood.
cbc/diff, lymphocytes, percent
poor: Less than 14%/good: 14 to 46%/poor: More than 46%
Lymphocytes are a type of white blood cell used by your immune system to protect against viral infections. The lymphocyte percent is the percentage of white blood cells that are lymphocytes. High amounts of lymphocytes can indicate viral infection. Zinc, along with vitamins B, and C are important to supporting a healthy immune system. Seafood, fruits and vegetables are good sources of zinc, vitamin B and vitamin C.
cbc/diff, monocytes, absolute count
poor: Less than 0.1×10E3/μL/good: 0.1 to 1.0×10E3/μL/poor: More than 1.0×10E3/μL
Monocytes are a type of white blood cell produced in your bone marrow. An immune system defense, they circulate through your blood vessels and protect your tissues against foreign objects. Monocytes are large cells, allowing them to swallow as much foreign material as possible. The monocyte absolute count is the actual number of monocytes present in your blood. High levels of monocytes can indicate infection. Zinc, along with vitamins B and C, are important to supporting a healthy immune system. Seafood, fruits and vegetables are good sources of zinc, vitamin B and vitamin C.
cbc/diff, metamyelocyte, percent
good: 0%/poor: More than 0%
Metamyelocytes are the forth cell type formed during the development of neutrophils, basophils, and eosinophils, which are the cells in your body that fight bacterial infection. The metamyelocyte percent is the percentage of white blood cells that are metamyelocytes. Metamyelocytes are typically found in your bone marrow and usually are not present in your blood. Your doctor can explain the significance of these cells in your blood.
cbc/diff, myelocyte, absolute count
good: 0.0×10E3/μL/poor: More than 0.0×10E3/μL
Myelocytes are the third cell type formed during the development of neutrophils, basophils, and eosinophils, which are the cells in your body that fight bacterial infection. The myelocyte absolute count is the actual number of myelocytes present in your blood. Your doctor can explain the significance of these cells in your blood.
cbc/diff, monocytes, percent
poor: Less than 4%/good: 4 to 13%/poor: More than 13%
Monocytes are a type of white blood cell produced in your bone marrow. An immune system defense, they circulate through your blood vessels and protect your tissues against foreign objects. Monocytes are large cells, allowing them to swallow as much foreign material as possible. The monocyte percent is the percentage of white blood cells that are monocytes. High levels of monocytes can indicate infection. Zinc, along with vitamins B and C, are important to supporting a healthy immune system. Seafood, fruits and vegetables are good sources of zinc, vitamin B, and vitamin C.
cbc/diff, neutrophils, absolute count
poor: Less than 1.8×10E3/μL/good: 1.8 to 7.8×10E3/μL/poor: More than 7.8×10E3/μL
Neutrophils are a type of white blood cell that fights bacterial infection. They are one of the first lines of defense your body uses against infections and inflammation. They migrate towards any inflammation in your body very rapidly. The neutrophil absolute count is the actual number of neutrophils present in your blood. High numbers of neutrophils can indicate the presence of infection or inflammation in your body.
cbc/diff, myelocyte, percent
good: 0%/poor: More than 0%
Myelocytes are the third cell type formed during the development of neutrophils, basophils, and eosinophils, which are the cells in your body that fight bacterial infection. The myelocyte percent is the percentage of white blood cells that are myelocytes. Your doctor can explain the significance of these cells in your blood.
cbc/diff, promyelocyte, absolute count
good: 0.0×10E3/μL/poor: More than 0.0×10E3/μL
Promyelocytes are the second cell type formed during the development of neutrophils, basophils, and eosinophils, which are the cells in your body that fight bacterial infection. The promyelocyte absolute count is the actual number of promyelocytes present in your blood. Your doctor can explain the significance of these cells in your blood.
cbc/diff, neutrophils, percent
poor: Less than 40%/good: 40 to 74%/poor: More than 74%
Neutrophils are a type of white blood cell that fights bacterial infection. They are one of the first lines of defense your body uses against infections and inflammation. They migrate towards any inflammation in your body very rapidly. The neutrophil percent is the percentage of white blood cells that are neutrophils. High numbers of neutrophils can indicate the presence of infection or inflammation in your body.
cbc/diff, promyelocyte, percent
good: 0%/poor: More than 0%
Promyelocytes are the second cell type formed during the development stages of neutrophils, basophils, and eosinophils, which are the cells in your body that fight bacterial infection. The promyelocyte percent is the percentage of white blood cells that are promyelocytes. Your doctor can explain the significance of these cells in your blood.
chloride
poor: Less than 97 mmol/L/good: 97 to 108 mmol/L/poor: More than 108 mmol/L
Chloride helps to maintain a balance in the amount of fluid inside and outside of your body's cells. It also aids in maintaining your body's pH or acid-base balance. Most of the chloride in your body comes from salt in your diet. High levels correlate to high levels of salt, a contributing factor to heart disease and high blood pressure. Increased levels can also be caused by certain medications or kidney disorders, since this organ controls the level of chloride. A chloride deficiency can be triggered by excessive fluid loss through sweating, vomiting or diarrhea.
cholesterol, total
excellent: Less than 100 mg/dL/good: 100 to 199 mg/dL/poor: More than 199 mg/dL
Cholesterol is a waxy fat-like substance, essential to good health, in the right amounts. Your body uses cholesterol to produce cell membranes, certain hormones, and bile, which aids in digestion. It has two main sources—75% is manufactured in the body and 25% comes from your food. Two main types of lipoproteins, HDL and LDL, carry cholesterol to and from your liver. LDL is known as the “bad” cholesterol—too much can increase your risk of heart disease. HDL is the “good” cholesterol and can protect your heart and arteries. Major dietary sources of cholesterol include egg yolks, beef, poultry, and shrimp. Obesity or being overweight raises your body's cholesterol levels.
creatinine
poor: Less than 0.57 mg/dL/good: 0.57 to 1.00 mg/dL/poor: More than 1.00 mg/dL
Creatinine is a chemical waste molecule generated from muscle metabolism in your body. Men tend to have higher levels than women because they have more skeletal muscle mass. Creatinine is transported to the kidneys, where it's filtered and disposed of through urine. Abnormally high levels can be indicative of kidney problems.
globulin, total
poor: Less than 1.5 g/dL/good: 1.5 to 4.5 g/dL/poor: More than 4.5 g/dL
Globulin is a protein comprised of antibodies and other proteins. Since elevated levels can be caused by chronic diseases and antibody deficiencies, globulin is a good indicator of immune health. Malnutrition, liver disease, kidney disease or severe trauma can cause a low globulin level.
gamma glutamyl transferase (ggt)
good: 0 to 60 IUnits/L/poor: More than 60 IUnits/L
Gamma Glutamyl Transferase (GGT) is an enzyme found on the surface of all the cells in your body. It's involved in the transfer of amino acids, the building blocks of protein, around your body. Chronic and acute alcohol abuse can cause high levels in the body. Elevated levels can also indicate abnormalities in the liver.
glomerular filtration rate, estimated (egfr)
poor: 59 mL/min/1.73 m² or less/good: More than 59 mL/min/1.73 m²
Estimated Glomerular Filtration Rate (eGFR) is a measurement that indicates your kidneys' flow rate of filtration or your kidneys' ability to remove waste products from your body. Levels can vary due to age, gender, race, and ethnicity, but eGFR is helpful in determining how your kidneys are functioning and the presence of kidney disease.
glucose
poor: Less than 65 mg/dL/good: 65 to 99 mg/dL/poor: More than 99 mg/dL
Glucose is an important sugar that acts as your body's main energy source. It is fueled by the consumption of carbohydrates. After eating, your blood glucose levels increase and then gradually decrease over time. The insulin secreted by your pancreas helps regulate your body's glucose level. Type II diabetics are either resistant to or do not produce enough insulin, which raises blood glucose levels and eventually causes an insulin deficiency.
ldl cholesterol, calculated
good: 0 to 99 mg/dL/poor: More than 99 mg/dL
LDL (Low Density Lipoprotein) Cholesterol is also known as “bad” cholesterol. High levels of LDL circulating in your blood can slowly build up in the inner walls of the arteries. Over time, this can form blockages in the arteries, possibly resulting in heart attack or stroke. Poor diets, often high in saturated fat, can cause elevated LDL levels in the blood.
hdl cholesterol
poor: Less than 40 mg/dL/good: 40 to 59 mg/dL/excellent: More than 59 mg/dL
HDL (High Density Lipoprotein) Cholesterol has been called “good” cholesterol because it appears to play a role in decreasing circulatory cholesterol. High HDL levels have been shown to reduce the development of coronary artery disease, while low levels can elevate your risk. Lifestyle is the best way to control HDL levels in your body. Exercise, weight loss, smoking cessation, and proper diet are some choices that can help control your HDL levels.
lipase, serum
good: 0 to 59 Units/L/poor: More than 59 Units/L
Lipase is an enzyme produced by your pancreas. Once produced, it is released into your small intestine to breakdown fat into fatty acids. Inflammation or damage to your pancreas or gall bladder and stomach or intestinal ulcers are common conditions that can increase the amount of lipase in your body.
ldl/hdl cholesterol ratio
good: 0.0 to 3.2/poor: More than 3.2
LDL/HDL Ratio measures the ratio of “bad” cholesterol to “good” cholesterol in your body. The higher your ratio, the higher your risk for coronary heart disease.
protein, total
poor: Less than 6.0 g/dL/good: 6.0 to 8.5 g/dL/poor: More than 8.5 g/d L
Total Protein is a measurement of the total amount of protein in your blood. The two major protein groups are albumin and globulins. Albumin is made in the liver and helps transport substances through the blood. It's important for tissue growth and healing. Globulins are produced by the liver and immune system. They function as transporters of metals in the blood and help fight infection. High total protein concentrations can be found in those with Hodgkin's lymphoma, leukemia, and plasma cell disorders. Low concentrations can indicate liver disease or infection.
potassium
poor: Less than 3.5 mmol/L/good: 3.5 to 5.2 mmol/L/poor: More than 5.2 mmol/L
Potassium is one of the most abundant minerals in your body. It's necessary for normal functioning of your cells, nerves and muscles, and for maintaining normal blood pressure levels. A balanced diet of fruits, vegetables, and meats will give you the proper amount of potassium. A shortage in your body can cause muscle weakness, slow reflexes, and heart problems.
sodium
poor: Less than 135 mmol/L/good: 135 to 145 mmol/L/poor: More than 145 mmol/L
Sodium, or salt, is an element essential for life. It's needed for the regulation of blood and body fluids, nerve impulses, and heart activity. Sodium balance is maintained by urination, sweating, and respiration. Too much salt in the body can cause hypernatremia, a rare and extreme form of dehydration. Hyponatremia, or too little sodium, can result in a dangerous condition known as water intoxication. The correct balance of sodium in your body is also important for avoiding conditions like high blood pressure and cardiovascular disease.
triglycerides
good: 0 to 149 mg/dL/poor: More than 149 mg/dL
Triglycerides are the form in which most fat exists in food and the body. They can be found in alcohol, saturated and trans fatty foods, and high carbohydrate foods and drinks. A normal amount of triglycerides in your bloodstream is good because it provides energy to your body. However, a high level of triglycerides caused by poor nutrition can increase your risk of heart disease, stroke, obesity, and diabetes.
vldl cholesterol, calculated
excellent: Less than 5 mg/dL/good: 5 to 40 mg/dL/poor: More than 40 mg/dL
VLDL (Very Low Density Lipoprotein) Cholesterol is one of the three major types of lipoproteins that move cholesterol and triglycerides throughout your body. Of these three lipoproteins, VLDL contains the most triglycerides. Like its counterpart LDL, elevated levels of this “bad” cholesterol can increase your risk for heart disease. Healthy lifestyle choices such as regular exercise and good eating habits can lower your triglyceride and VLDL levels.

Some example reports may be presented to a patient electronically (such as via a secure web site) and/or in hard copy (such as by mail, in person in a health provider's office, or by printing a downloaded electronic copy).

Some example reports may contain additional pages, such as general information pages which may provide general health information. For example, a general information page may include the following text:
myths about metabolism
1. Carrying extra weight makes your metabolism slower. Actually, extra weight causes your body to work harder to sustain itself. That's why it's easier to lose weight at the start of a diet, but harder later on.
2. Problems with metabolism are often related to the thyroid. Although defects in the thyroid gland can slow metabolism, this problem is rare. Some people just burn calories at a slower rate than others.
3. Eating less helps you lose more weight. Small and frequent meals keep your metabolism up, burning more calories overall. Too much time between meals causes your body to hoard rather than burn calories, slowing your metabolism.
4. Aerobic exercise is best for increasing your metabolism. 30 minutes of aerobic exercise burns more calories than 30 minutes of weight training. But in the long run, strength training builds the muscle that burns fat and boosts metabolism.
5. Certain foods burn more fat than others. Some studies have shown that green tea, red pepper and very spicy foods can raise metabolism for a short time. But, the impact is slight when you're trying to lose weight. Your best plan: small, frequent meals and low-calorie, high protein snacks.
more information
on the web
www.mayoclinic.com/health/metabolism/WT00006
www.consumer.gov/weightloss
www.webmd.com/diet
in print
Healthy Weight for Every Body
Mayo Clinic
support
www.3fatchicks.com
cholesterol—is it a cause or result of being overweight?
Cholesterol is a molecule that has gotten a bad rap. This waxy, fat-like substance exists naturally throughout your body and is needed for good health. It's used to produce cell membranes, hormones and bile, which aids in digestion. Cholesterol itself does not increase the amount of fat in your body. High cholesterol is a consequence, not a cause.
Obesity or being overweight raises your cholesterol levels. Even more concerning is that people who are overweight have shown lower levels of high density lipoprotein (HDL) and normal to high levels of low density lipoprotein (LDL). These lipoproteins are responsible for transporting cholesterol around your body. High levels of HDL have been associated with lowering heart disease risk, while high levels of LDL have been linked to an increased risk. Excess weight also raises the levels of triglycerides or fat in your body.
So, instead of lowering cholesterol to reduce your weight, you should be reducing your weight to lower your cholesterol! The type of fats you eat (not high cholesterol foods) has the greatest effect on your blood cholesterol levels. Avoid a diet high in saturated and trans fats and aim to get the right amount of daily calories for your body. Exercise is the best way to expend any excess calories.
Excess fat not only raises your cholesterol levels, but increases your risk of diabetes, high blood pressure and heart disease. Your best bet for good health? Proper diet and exercise.
Metabolism: by the numbers
35 calories The number of calories burned by each pound of muscle in your body—fat burns only two calories per day
5 percent The rate at which metabolism slows per decade after age 40 133.6 million The number of US adults who are overweight or obese 25 bmi Overweight as defined by the World Health Organization (a body mass index of more than 25)
$487 billion The savings on medical, fuel, food, and other costs if Americans were not overweight—that's enough to give every US household more than $4,000 (source: What if no one were fat? MSN.com, April 2008)

Lifestyle Assessment

An example lifestyle assessment according to the present disclosure may include an instruction page. An example instruction page may include the following text:
how to read this assessment
Your Viveda Lifestyle assessment provides a tremendous amount of information on your health. Where should you start? Following these steps can help you and your physician understand your assessment:
1. Start with the summary page. The summary page offers an overview of your assessment results, including:
   Your level of health risk
   Important numbers gathered at the time of your blood test
   Your number of in balance (good and excellent) and out of balance (poor) readings
   The top 5 molecule readings that require your priority attention
2. Understand your detailed Lifestyle bioprofile. This information will help you understand how we determined your health risk, your top 5 priority molecule readings, and your in and out of balance readings. You'll see how we divided your 68 molecular measurements into priority categories and whether you received a poor, good, or excellent reading for each molecule.
3. Review your individual readings. Your readings are divided into two parts—in balance and out of balance. The in balance readings include molecules with good or excellent readings and out of balance readings are molecules with poor readings. Spend some time reviewing the definitions for your out of balance readings and discuss each molecule's significance with your doctor. Pay particular attention to your top 5 priority molecules.
4. Use your resources. Use the tips and information on the resources pages to increase your understanding and improve your health.
5. Develop your plan. Now that you know more about your health, you can make informed decisions that help you prevent illness and achieve a balanced, healthy life. Work with your doctor to develop a wellness plan tailored just for you. It's important to develop strategies that you can implement. For example, an hour of exercise every day may not be realistic for you. Your physician is your partner. Collaborate to identify realistic goals and steps that set you up for success.

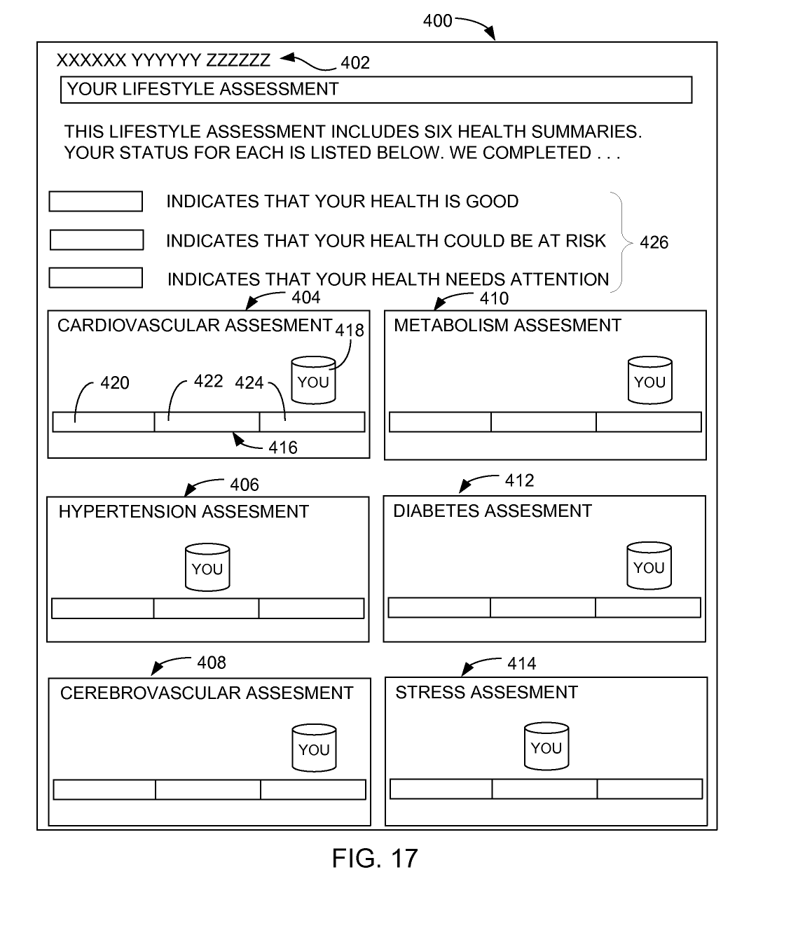

An example lifestyle assessment may include a summary page 400 as illustrated in FIG. 17. An example summary page 400 may include patient identifying information 402. An example summary page 400 may include summary graphical representations of various health areas, such as cardiovascular summary 404, hypertension summary 406, cerebrovascular summary 408, metabolism summary 410, diabetes summary 412, and/or stress summary 414. Individual summaries 404, 406, 408, 410, 412, 414 may include a scale 416 on which a marker 418 is located, reflecting the patient's status. An example scale 416 may include a portion 420 associated with a “good” categorization, a portion 422 associated with an “at risk” categorization, and/or a portion 424 associated with a “needs attention” categorization. An example summary page 400 may include a legend 426, which may aid a reader in interpreting the scales 416. An example summary page may include the following text:
This lifestyle assessment includes six health summaries. Your status for each is listed below. We completed more than 270 molecular measurements on your blood sample to determine your current health status in each area. Working with your physician, use this information to decide which area of health you should focus on first. Inside this report, you'll find detailed information for each of the six areas listed below, including tips on how to improve your results.

An example lifestyle assessment may include a page describing a bioprofile, which may include the following text:
about your bioprofile
Using the blood sample taken by your physician's office, 68 molecular measurements have been completed for your Viveda Lifestyle assessment. These molecules tell a story about what's happening inside your body. A story that is critical to understanding and managing your health and wellness. When you know more about your health, you can make informed, proactive decisions that help you prevent illness and enjoy life today and tomorrow.
what is a bioprofile?
This assessment provides a detailed account of your Lifestyle bioprofile. In scientific terms, a bioprofile is an analysis of your body at the molecular level. In medical terms, a bioprofile is an overview of your current health condition and your risk for disease.
what are the steps for determining my bioprofile?
1. An area of health is chosen for evaluation. Let's use cardiovascular health as our example.
2. We determine the list of molecules to be analyzed. An enormous body of evidence based medical knowledge amassed over the last 60 years is searched to determine that list. We choose the molecules that are the best indicators of cardiovascular health.
3. The targeted cardiovascular molecules are linked together within their biological network, establishing the function and importance of each molecule to cardiovascular health. At this point, we also determine the priority of each molecule to cardiovascular health and the acceptable, healthy range of that molecule in an individual's body.
4. Using our scientific instrumentation we analyze your blood sample, measuring the targeted list of molecules. These measurements are compiled to produce your cardiovascular bioprofile.
5. You and your physician receive your bioprofile results in the Viveda Health and Wellness Assessment.
what happens next?
Work with your doctor and use this information to develop your own health and wellness plan. You can make informed, proactive decisions about your health, instead of waiting until something goes wrong or you don't feel well. Viveda can help you identify problem areas, before you even know there is a problem.
After you've had a chance to implement your wellness plan, use Viveda again to discover if you have improved your health. In six months to a year, repeat your Viveda assessment to see if you have improved your bioprofile readings. It's fun to compete against yourself and it's empowering to know that you've taken control of your own health. The goal of our partnership—Viveda, your physician, and you—is to improve your quality of life. It isn't just about living longer, but living better and healthier. Know your health and plan for a healthy future!

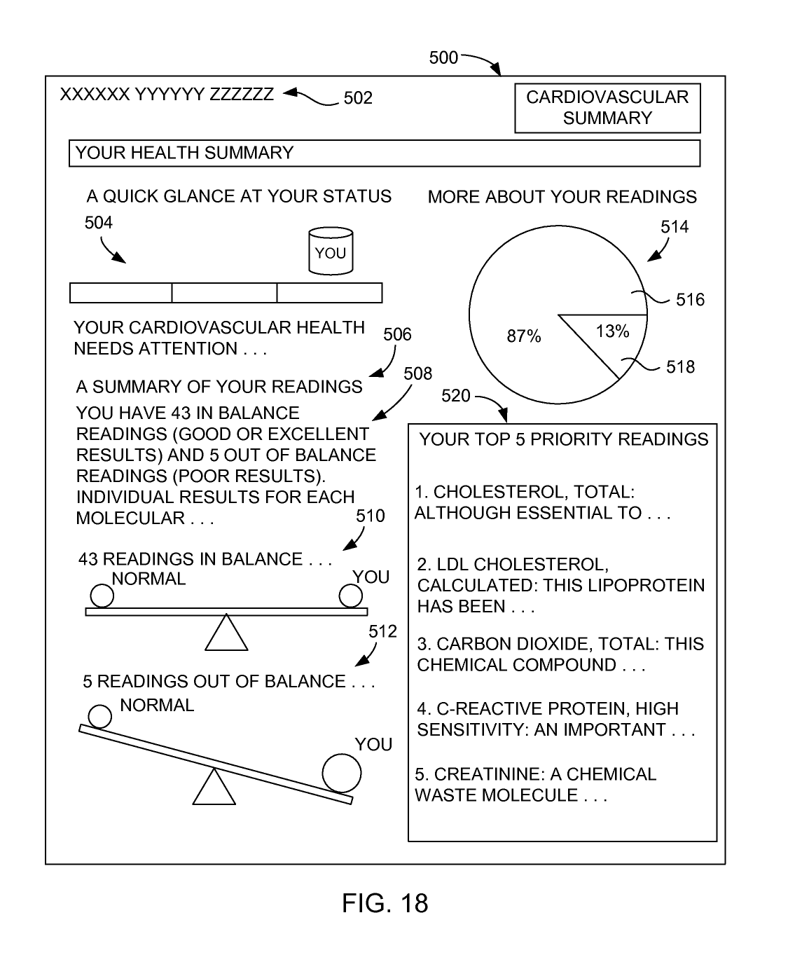

An example lifestyle assessment may include a cardiovascular section including a cardiovascular summary page 500 as illustrated in FIG. 18. An example cardiovascular summary page 500 may include patient identifying information 502. An example cardiovascular summary page 500 may include a graphical representation 504 of the cardiovascular health status of the patient, similar to cardiovascular summary 404 on summary page 400. The graphical representation 504 may be accompanied by appropriate text, such as the following:
Your cardiovascular health needs attention, you may be at risk for a heart attack. Talk with your physician today about ways to improve poor readings for critical and extremely important molecules (see your bioprofile on the next page).

A summary section 506 may include a textual summary 508 of the readings related to cardiovascular health. For example, the textual summary 508 may include the following:
You have 43 in balance readings (good or excellent results) and 5 out of balance readings (poor results). Individual results for each molecular measurement are in the cardiovascular readings section of this assessment.

An example summary section 506 may include a graphical representation 510 of the in balance readings. For example, the graphical representation 510 may include a figure showing a balance in a balanced configuration. An example summary section 506 may include a graphical representation 512 of the out of balance readings. For example, the graphical representation 512 may include a figure showing a balance in an out of balance configuration.

An example cardiovascular summary page 500 may include a graphical representation showing the percentages of the readings relevant to cardiovascular health that fall into each of a plurality of categories. For example, a pie chart 514 may indicate a percentage of readings that are categorized as “good” 516 and/or a percentage of readings that are categorized as “poor” 518. In some example embodiments, such a pie chart 514 may include a portion representing a percentage of readings that are categorized as “excellent.”

An example cardiovascular summary page 500 may include a priority readings section 520, which may identify one or more readings that may have a higher priority for action by the patient. For example, a priority readings section 520 may include the following text:
your top 5 priority readings
48 molecular measurements were completed for your assessment. The molecules below represent your most critical results and should be given priority attention by you and your physician.
1. Cholesterol, Total: Although essential to good health, excess cholesterol is linked to heart disease, stroke, and obesity. Excess weight caused by a high fat diet and lack of exercise can raise your cholesterol.
2. LDL Cholesterol, Calculated: This lipoprotein has been shown to cause plaque build-up in arteries at high levels. A diet low in saturated fats and adequate exercise can lower your LDL amounts.
3. Carbon Dioxide, Total: This chemical compound helps maintain healthy levels of acidity and other fluids in your body. Acidity levels can be affected by foods, medications and kidney and lung function.
4. C-Reactive Protein, High Sensitivity: An important indicator of cardiovascular and stroke risk, high CRP levels can result from inflammation. Poor diet is a key contributor to inflammation in your body.
5. Creatinine: A chemical waste molecule generated by muscle metabolism, creatinine is a good indicator of kidney function.

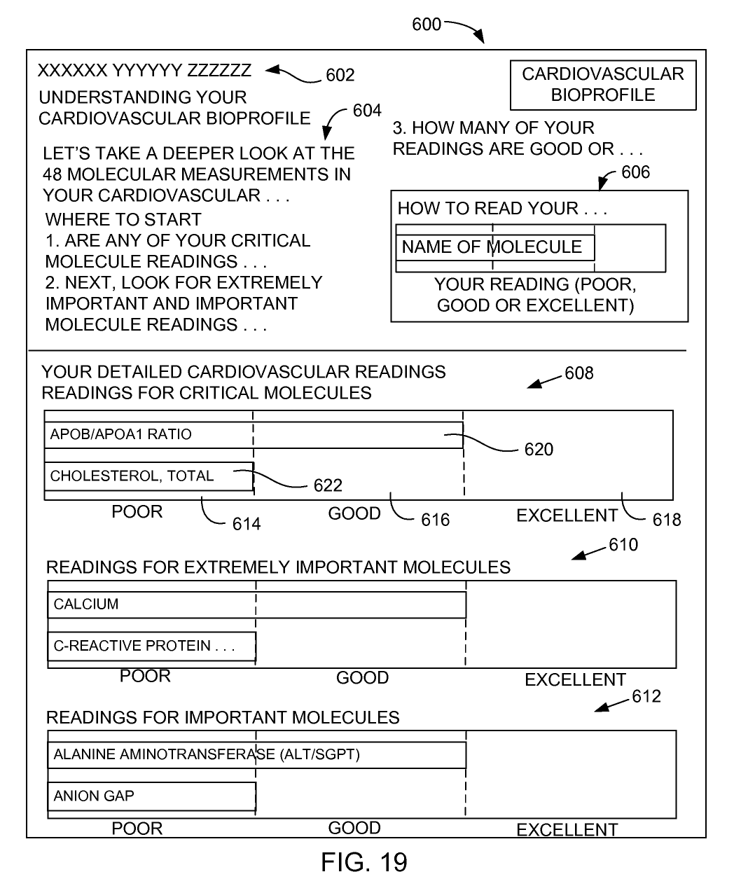

An example lifestyle assessment may include a cardiovascular bioprofile page 600 as illustrated in FIG. 19. An example cardiovascular bioprofile page 600 may include identifying information 602. An example cardiovascular bioprofile page 600 may include information related to the bioprofile, such as the following text:
Let's take a deeper look at the 48 molecular measurements in your cardiovascular bioprofile. Your bioprofile is based on two levels of information. First is the priority of each molecule or its importance to your health. Molecules are critical, extremely important or important. Next, is the amount of the molecule in your body. Your molecule readings are categorized as poor, good, or excellent as compared to an acceptable, healthy range.
where to start
1. Are any of your critical molecule readings red? Focus your efforts on improving these poor readings first.
2. Next, look for extremely important and important molecule readings that are red. Though not as critical, these molecules are still important to your health.
3. How many of your readings are good or excellent? Think about positive lifestyle habits that help you maintain these readings.

Some example cardiovascular bioprofile pages 600 may include graphical representations of individual readings. Some example cardiovascular bioprofile pages 600 may include graphical representations of individual readings grouped according to the importance of the respective molecules. For example, an example cardiovascular bioprofile page 600 may include a critical molecules section 608, an extremely important molecules section 610, and/or an important molecules section 612. Individual sections 608, 610, 612 may include vertical columns 614, 616, 618 associated with poor, good, and excellent categorizations, respectively. Individual readings may be represented as horizontal bars 620, 622, which may extend across columns 614, 616, 618 to reflect the categorization of the individual readings. For example, a reading for APOB/APOA1 ratio may be categorized as good, and the respective horizontal bar 620 may extend across the poor column 614 and the good column 616. An example cardiovascular bioprofile page 600 may include instructions 606 for reading graphical portions of the bioprofile page 600.

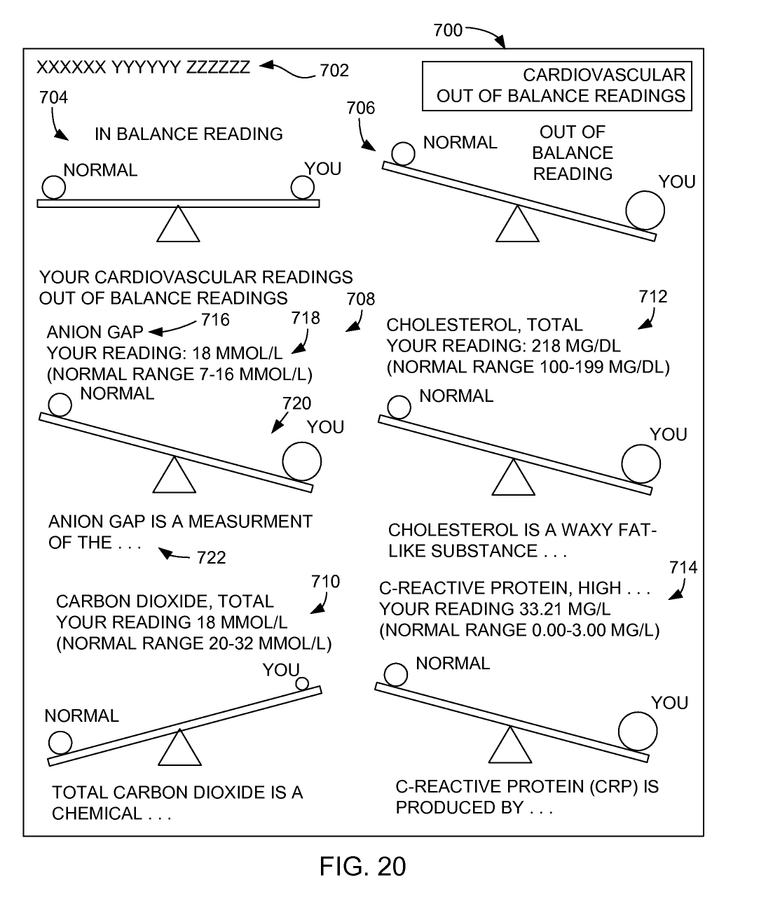

An example lifestyle assessment may include a cardiovascular out of balance readings page 700 as illustrated in FIG. 20. An example cardiovascular out of balance readings page 700 may include identifying information 702 and/or the following text:
understanding your cardiovascular readings
Your individual molecule readings are listed below. The in balance readings include good or excellent readings that fell into the acceptable, healthy range. Out of balance or poor readings include molecules outside of the acceptable, healthy range. If you are above normal, the ball on the right is bigger and tips the balance down. A reading that is below normal makes the ball smaller and tips the balance up.

An example cardiovascular out of balance readings page 700 may include a legend, which may include an example graphical representation 704 of an in balance reading and/or an example graphical representation 706 of an out of balance reading.

An example cardiovascular out of balance readings page 700 may include sections 708, 710, 712, 714 associated with individual out of balance readings. Individual sections 708, 710, 712, 714 may include the name of the molecule 716, a listing of the measured value and the normal range for the molecule 718, a graphical representation of the reading compared to the normal range (e.g., a balance), and/or text 722 pertaining to the molecule. An example out of balance reading page 700 may include the following text:
Poor readings that are outside of the acceptable, healthy range.
anion gap
Your reading: 18 mmol/L (normal range 7-16 mmol/L)
Anion Gap is a measurement of the positively and negatively charged ions in your blood. Sodium and potassium, known as cations, are positively charged. Chloride and bicarbonate, called anions, are negatively charged. To maintain optimal health, your body requires a balance between cations and anions.
carbon dioxide, total
Your reading: 18 mmol/L (normal range 20-32 mmol/L)
Total Carbon Dioxide is a chemical compound that acts as a buffer to maintain normal levels of acidity (pH) in blood and other fluids in your body. Your body's acidity can be affected by foods or medication and by kidney and lung function. High levels can indicate chronic obstructive pulmonary disease (COPD), fluid in the lungs and heart disease. Hyperventilation, liver or kidney disease, hyperthyroidism, or uncontrolled diabetes can result in low levels of bicarbonate, a component of carbon dioxide.
cholesterol, total
Your reading: 218 mg/dL (normal range 100-199 mg/dL)
Cholesterol is a waxy fat-like substance, essential to good health, in the right amounts. Your body uses cholesterol to produce cell membranes, certain hormones, and bile, which aids in digestion. It has two main sources—75% is manufactured in the body and 25% comes from your food. Two main types of lipoproteins, HDL and LDL, carry cholesterol to and from your liver. LDL is known as the “bad” cholesterol—too much can increase your risk of heart disease. HDL is the “good” cholesterol and can protect your heart and arteries. Major dietary sources of cholesterol include egg yolks, beef, poultry, and shrimp. Obesity or being overweight raises your body's cholesterol levels.
c-reactive protein, high sensitivity
Your reading: 33.21 mg/L (normal range 0.00-3.00 mg/L)
C-Reactive Protein (CRP) is produced by your liver. CRP levels increase dramatically during the inflammatory process in an effort to detoxify the body of substances released from damaged tissue. Elevated levels can be seen in people with inflammatory diseases, liver diseases, and severe infections. CRP has also been identified as an important indicator of poor cardiovascular health. A CRP test is often used by physicians to test for heart failure.
ldl cholesterol, calculated
Your reading: 137 mg/dL (normal range 0-99 mg/dL)
LDL (Low Density Lipoprotein) Cholesterol is also known as “bad” cholesterol. High levels of LDL circulating in your blood can slowly build up in the inner walls of the arteries. Over time, this can form blockages in the arteries, possibly resulting in heart attack or stroke. Poor diets, often high in saturated fat, can cause elevated LDL levels in the blood.

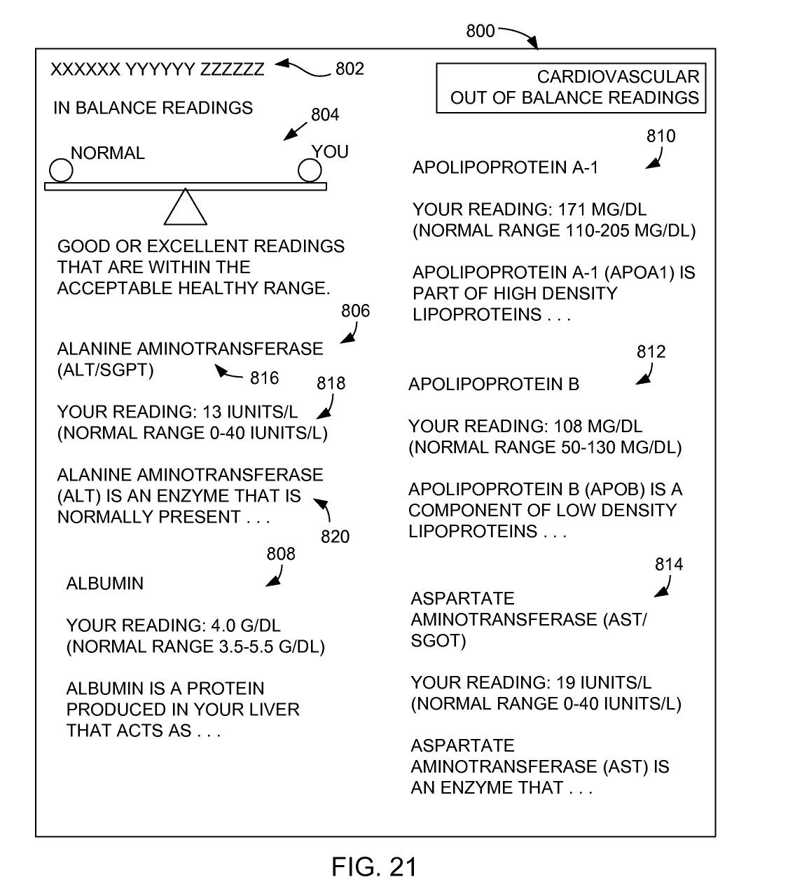

An example lifestyle assessment may include a cardiovascular in balance readings page 800 as illustrated in FIG. 21. An example cardiovascular in balance readings page 800 may include identifying information 802 and/or a graphical depiction 804 of an in balance reading (e.g., a balance). An example cardiovascular in balance readings page 800 may include sections 806, 808, 810, 812, 814 associated with in balance readings. Individual sections 806, 808, 810, 812, 814 may include the name of the molecule 816, a listing of the measured value and the normal range for the molecule 818, and/or text 820 pertaining to the molecule. An example out of balance reading page 800 may include the following text:
Good or excellent readings that are within the acceptable, healthy range.
alanine aminotransferase (alt/sgpt)
Your reading: 13 IUnits/L (normal range 0-40 (Units/L)
Alanine Aminotransferase (ALT) is an enzyme that is normally present at low levels in your body. Increased amounts of ALT in your body can be a result of liver or heart damage. Some medications also elevate ALT levels. This enzyme is an important indicator of liver health.
albumin
Your reading: 4.0 g/dL (normal range 3.5-5.5 g/dL)
Albumin is a protein produced in your liver that acts as a transporter. It transports essential fatty acids to your muscles and moves hormones, drugs, and other small molecules through your blood. The most abundant protein in your blood, it also keeps the fluid in your blood from leaking into tissue. Decreased levels can indicate problems with your kidney or liver, infection, malnutrition or the effects of a low-protein diet. Eating high-protein foods can help increase the amount in your body.
albumin/globulin (a/g) ratio
Your reading: 1.4 (normal range 1.1-2.5)
Albumin/Globulin Ratio is a measurement that provides information on your body's nutritional status. It's also used to evaluate kidney and liver health. When albumin levels drop, your body increases globulin levels in an effort to maintain a normal total protein level. An abnormal albumin/globulin ratio indicates an imbalance of the types of proteins present in your blood.
alkaline phosphatase
Your reading: 96 IUnits/L (normal range 25-150 (Units/L)
Alkaline Phosphatase (ALP) is an enzyme produced by your liver; but it is also made by your bones, intestines, and kidneys. It is released by your body into the blood during injury and normal activities such as bone growth and pregnancy. Your physician uses it to assess your liver health. Bone or liver disease, heart problems, infection, alcohol consumption, and certain medications can raise the amounts in your body.
apob/apoa1 ratio
Your reading: 0.6 (normal range 0.0-0.6)
APO B/APO A 1 Ratio is a measurement used to assess your cardiovascular risk. This ratio indicates the balance between “good” and “bad” cholesterol in your body.
apolipoprotein a-1
Your reading: 171 mg/dL (normal range 110-205 mg/dL)
Apolipoprotein A-1 (APOA1) is a part of high density lipoproteins (HDL or “good” cholesterol). One of its many purposes is to help clear and prevent the build-up of cholesterol in the arteries.
apolipoprotein b
Your reading: 108 mg/dL (normal range 50-130 mg/dL)
Apolipoprotein B (APOB) is a component of low density lipoproteins (LDL or “bad cholesterol”), responsible for carrying cholesterol to tissues. It “unlocks” the doors to cells and delivers cholesterol to them. High levels of APOB can lead to the buildup of plaque, which can cause atherosclerosis.
apolipoprotein e
Your reading: 4 mg/dL (normal range 3-7 mg/dL)
Apolipoprotein E (APOE), a protein produced by your liver, is responsible for transporting lipoproteins, fat-soluble vitamins, and cholesterol in the blood. Elevated levels have been linked to cardiovascular disease and problems with lipoprotein balance.
aspartate aminotransferase (ast/sgot)
Your reading: 19 IUnits/L (normal range 0-40 IUnits/L)
Aspartate Aminotransferase (AST) is an enzyme that naturally circulates in your body. It can be found in your red blood cells, liver, heart, muscle tissue, pancreas, and kidneys. Normally present in a low amount, damage or disease in the heart and liver can increase AST levels. Medications and excessive alcohol use can also increase the amount of AST in your body.
bilirubin, total
Your reading: 0.3 mg/dL (normal range 0.1-1.2 mg/dL)
Bilirubin is a compound found in bile that is produced when the liver breaks down old red blood cells. When the body overproduces bilirubin or the liver cannot process it, jaundice occurs. Your physician uses bilirubin concentration to determine whether your liver is functioning properly.
blood urea nitrogen
Your reading: 15 mg/dL (normal range 5-26 mg/dL)
Blood Urea Nitrogen (BUN) is a test routinely used to evaluate kidney function. Urea nitrogen is a waste product of protein metabolism found in your blood. Urea is formed by the liver and carried through your bloodstream to the kidneys for excretion. Increased levels in a BUN test may also indicate dehydration, stress, excessive protein intake or even heart problems. Decreased levels are rare, but could be caused by pregnancy, anabolic steroid use, low protein diet, malnutrition or liver disease.
bun/creatinine ratio
Your reading: 19 (normal range 8-27)
BUN/Creatinine Ratio is a comparison between your BUN (Blood Urea Nitrogen) level and creatinine level. Physicians often use this ratio to check for kidney damage, dehydration or problems with kidney function.
calcium
Your reading: 9.3 mg/dL (normal range 8.5-10.6 mg/dL)
Calcium (Ca2+) is the most abundant mineral in your body. More than 99% of the calcium in your body is stored in the bones and teeth. Calcium is crucial to building and maintaining strong bones, but it also serves other important functions. It allows your blood to clot, muscles to contract, nerves to send messages, and your brain to process information. Studies have also shown that calcium contributes to healthy blood pressure. Your body gets the calcium it needs in two ways. The most important method is through consumption of calcium-rich foods including dairy products and dark, leafy greens. If your diet is calcium deficient your body will get what it needs from your bones, which can weaken bone structure.
catecholamines, total plasma
Your reading: <398 pg/mL (normal range 0-642 pg/mL)
Catecholamines are commonly referred to as ‘stress’ hormones because they are responsible for your body's fight-or-flight response. Released by the adrenal gland during stressful situations they increase your heart rate, blood pressure, respiration rate, muscle strength, and mental alertness. Prolonged, excessive release from chronic stress or a tumor can result in hypertension. High levels can also indicate anxiety, stress, thyroid disorders, hypoglycemia, heart disease or an adrenal gland tumor.
chloride
Your reading: 105 mmol/L (normal range 97-108 mmol/L)

Chloride helps to maintain a balance in the amount of fluid inside and outside of your body's cells. It also aids in maintaining your body's pH or acid-base balance. Most of the chloride in your body comes from salt in your diet. High levels correlate to high levels of salt, a contributing factor to heart disease and high blood pressure. Increased levels can also be caused by certain medications or kidney disorders, since this organ controls the level of chloride. A chloride deficiency can be triggered by excessive fluid loss through sweating, vomiting or diarrhea.
creatinine
Your reading: 0.80 mg/dL (normal range 0.57-1.00 mg/dL)
Creatinine is a chemical waste molecule generated from muscle metabolism in your body. Men tend to have higher levels than women because they have more skeletal muscle mass. Creatinine is transported to the kidneys, where it's filtered and disposed of through urine. Abnormally high levels can be indicative of kidney problems.
dopamine
Your reading: <10 pg/mL (normal range 0-142 pg/mL)
Dopamine is an important molecule that serves as a neurotransmitter and hormone. In the brain, it has roles in controlling behavior, cognition, sleep, mood, and motor activity. It's also released by the body according to the amount of pain a person is experiencing. Low levels of dopamine can cause social anxiety, and have been linked to attention-deficit hyperactivity disorder (ADHD). Abnormally high levels have been linked to psychosis, schizophrenia, and can cause hypersociality and hypersexuality in manic depressive patients.
epinephrine
Your reading: 19 pg/mL (normal range 0-99 pg/mL)
Epinephrine, also known as adrenalin, is a naturally occurring hormone. During the “fight-or-flight” stress response, it's released into the bloodstream to increase heart rate, blood pressure and blood flow to the muscles. A deficiency can lead to problems with conversion of sugars. High levels can lead to elevated blood pressure, difficulty breathing, anxiety, confusion, and an irregular heartbeat.
estradiol
Your reading: 34 pg/mL (normal range 19-528 pg/mL)
Estradiol is a potent naturally occurring estrogen hormone. Present in both men and women, the highest levels of this sex hormone are seen in pre-menopausal females. Critical for proper sexual function, estradiol is necessary for healthy bones. It is also used to treat symptoms of menopause, hypertension, to prevent osteoporosis, and in some cancer treatments. Research indicates that estradiol could be helpful in reducing cardiovascular risk.
ferritin
Your reading: 28 ng/mL (normal range 10-291 ng/mL)
Ferritin is a protein that stores iron in your body for later use. The amount of iron in your blood is directly related to the amount of ferritin in your body. High levels have been linked to blood disorders and inflammation. Low levels can be a result of iron deficiency, long term bleeding or heavy menstruation in women.
folate (vitamin b9)
Your reading: 20.6 ng/mL (normal range>5.4 ng/mL)
Folate (Vitamin B9) is extremely important to the production and maintenance of new cells. Critical in early pregnancy, folic acid helps prevent birth defects and premature births. Leafy green vegetables, whole grains, lean meats, legumes, and beans are good food sources. Too much folic acid rarely causes harm, but too little can lead to certain types of anemia.
globulin, total
Your reading: 2.8 g/dL (normal range 1.5-4.5 g/dL)
Globulin is a protein comprised of antibodies and other proteins. Since elevated levels can be caused by chronic diseases and antibody deficiencies, globulin is a good indicator of immune health. Malnutrition, liver disease, kidney disease or severe trauma can cause a low globulin level.
glomerular filtration rate, estimated (egfr)
Your reading: >59 mL/min/1.73 m² (normal range>59 mL/min/1.73 m²)
Estimated Glomerular Filtration Rate (eGFR) is a measurement that indicates your kidneys' flow rate of filtration or your kidneys' ability to remove waste products from your body. Levels can vary due to age, gender, race, and ethnicity, but eGFR is helpful in determining how your kidneys are functioning and the presence of kidney disease.
glucose
Your reading: 93 mg/dL (normal range 65-99 mg/dL)
Glucose is an important sugar that acts as your body's main energy source. It is fueled by the consumption of carbohydrates. After eating, your blood glucose levels increase and then gradually decrease over time. The insulin secreted by your pancreas helps regulate your body's glucose level. Type II diabetics are either resistant to or do not produce enough insulin, which raises blood glucose levels and eventually causes an insulin deficiency.
hdl cholesterol
Your reading: 53 mg/dL (normal range 40-59 mg/dL)
HDL (High Density Lipoprotein) Cholesterol has been called “good” cholesterol because it appears to play a role in decreasing circulatory cholesterol. High HDL levels have been shown to reduce the development of coronary artery disease, while low levels can elevate your risk. Lifestyle is the best way to control HDL levels in your body. Exercise, weight loss, smoking cessation, and proper diet are some choices that can help control your HDL levels.
homocyst(e)ine, serum/plasma
Your reading: 5.9 μmol/L (normal range 0.0-15.0 μmol/L)
Homocysteine is an amino acid produced in your body, but also acquired when you eat meat. Levels can be influenced by genetic factors and diet. Foods or supplements that contain folic acid, vitamin B6, and vitamin B12 are effective in lowering high homocysteine levels. High levels are related to an increased risk of coronary heart disease, stroke, and vascular disease.
il-10
Your reading: 0.4 pg/mL (normal range 0.0-14.4 pg/mL)
Interleukin-10 (IL-10) belongs to a group of proteins that function as chemical messengers between cells. IL-10 regulates the immune system's response by controlling inflammation. A high level of IL-10 indicates a high level of inflammation in the body, which is indicative of poor health or illness.
il-6
Your reading: <2.0 pg/mL (normal range 0.0-14.0 pg/mL)
Interleukin-6 (IL-6) is a protein that improves your body's natural response to infection and disease. When your body experiences trauma such as burns, infection or tissue damage, nearby cells secrete IL-6 which results in inflammation. Elevated amounts of IL-6 have been associated with heart attack, diabetes, and stroke.
insulin
Your reading: 9.0 μUnits/mL (normal range 0.0-29.1 μUnits/mL)
Insulin is a hormone produced by your pancreas to help turn sugar (glucose) from your food into energy. It is a critical component to body function. Without insulin, you could eat and still starve because your cells couldn't access the glucose in your bloodstream.
iron
Your reading: 106 μg/dL (normal range 35-155 μg/dL)
Iron is an element that is essential to all life. It plays a key role in transporting oxygen and carbon dioxide throughout your body. Iron must be consumed through diet and is found in foods such as red meat, beans, and green leafy vegetables. Too much iron can interfere with nutrient absorption and can cause organ damage over time.
ldl/hdl cholesterol ratio
Your reading: 2.6 (normal range 0.0-3.2)
LDL/HDL Ratio measures the ratio of “bad” cholesterol to “good” cholesterol in your body. The higher your ratio, the higher your risk for coronary heart disease.
norepinephrine
Your reading: 369 pg/mL (normal range 0-399 pg/mL)
Norepinephrine is a chemical that functions as a hormone and a neurotransmitter. It is the building block to the more well-known molecule adrenaline. As a hormone, it's released in stressful situations, causing your heart rate to quicken, increasing blood flow to muscles, and increasing attention. This reaction is often called the “fight-or-flight” response. Increased levels can result in hypertension. A deficiency can cause attention disorders and depression.
phosphorus
Your reading: 3.3 mg/dL (normal range 2.5-4.5 mg/dL)
Phosphorus is a chemical element essential for all living cells. It's a component of DNA and RNA and is used for energy transport among cells. A balanced diet that includes meat and milk provides sufficient phosphorous intake. Deficiency is rare, but symptoms can include anemia, bone problems, and neurological problems. High levels can lead to calcium deposits in the muscles.
potassium
Your reading: 3.9 mmol/L (normal range 3.5-5.2 mmol/L)
Potassium is one of the most abundant minerals in your body. It's necessary for normal functioning of your cells, nerves and muscles, and for maintaining normal blood pressure levels. A balanced diet of fruits, vegetables, and meats will give you the proper amount of potassium. A shortage in your body can cause muscle weakness, slow reflexes, and heart problems.
protein, total
Your reading: 6.8 g/dL (normal range 6.0-8.5 g/dL)
Total Protein is a measurement of the total amount of protein in your blood. The two major protein groups are albumin and globulins. Albumin is made in the liver and helps transport substances through the blood. It's important for tissue growth and healing. Globulins are produced by the liver and immune system. They function as transporters of metals in the blood and help fight infection. High total protein concentrations can be found in those with Hodgkin's lymphoma, leukemia, and plasma cell disorders. Low concentrations can indicate liver disease or infection.
sodium
Your reading: 141 mmol/L (normal range 135-145 mmol/L)
Sodium, or salt, is an element essential for life. It's needed for the regulation of blood and body fluids, nerve impulses, and heart activity. Sodium balance is maintained by urination, sweating, and respiration. Too much salt in the body can cause hypernatremia, a rare and extreme form of dehydration. Hyponatremia, or too little sodium, can result in a dangerous condition known as water intoxication. The correct balance of sodium in your body is also important for avoiding conditions like high blood pressure and cardiovascular disease.
testosterone, total
Your reading: 24 ng/dL (normal range 14-76 ng/dL)
Testosterone is a steroid hormone that is essential to the well being of both men and women. The male body produces considerably more testosterone, which is the principal male sex hormone. However, it plays a key role in the health of both genders affecting libido, energy, mood, fertility, and bone strength.
triglycerides
Your reading: 142 mg/dL (normal range 0-149 mg/dL)
Triglycerides are the form in which most fat exists in food and the body. They can be found in alcohol, saturated and trans fatty foods, and high carbohydrate foods and drinks. A normal amount of triglycerides in your bloodstream is good because it provides energy to your body. However, a high level of triglycerides caused by poor nutrition can increase your risk of heart disease, stroke, obesity, and diabetes.
troponin i
Your reading: <0.2 ng/mL (normal range 0.0-0.4 ng/mL)
Troponin is a family of proteins needed for skeletal and cardiac muscle contraction. Troponin I and T are both good indicators of heart inflammation, and often used by physicians to diagnose a heart attack and determine the extent of heart muscle damage. Troponin C is used by skeletal muscle to induce muscle contraction. Congestive heart failure, severe infections, kidney disease, and heart inflammation may cause elevated troponin levels.
tumor necrosis factor
Your reading: 6.2 pg/mL (normal range 0.0-8.1 pg/mL)
Tumor Necrosis Factor is a protein made by white blood cells to stimulate the immune system in response to infection or disease. TNF is a good indicator of inflammation in your body, caused by poor health or illness.
uric acid
Your reading: 5.8 mg/dL (normal range 2.4-8.2 mg/dL)
Uric Acid is a waste product from the breakdown of substances consumed through your diet. The majority of it is filtered by your kidneys and excreted through urine. Obesity, excessive drinking, medications, and diet can cause uric acid to accumulate. A build-up can cause the painful condition of gout as well as kidney stones, kidney damage, and arthritis.
vitamin c
Your reading: 0.5 mg/dL (normal range 0.4-2.0 mg/dL)
Vitamin C is a water soluble vitamin and antioxidant needed for healthy bones, blood vessels, and skin. Your body doesn't manufacture vitamin C and, because it dissolves in water, cannot store it. A continuous supply is needed from your daily diet. Vitamin C helps produce collagen, a protein used to make skin, scar tissue, tendons, ligaments, and blood vessels. It promotes the healing of wounds and burns, helps fight infection and blocks some of the damage caused by free radicals in your body. Research shows that vitamin C can also help decrease your chance of heart disease. Important dietary sources for this essential nutrient include vegetables and fruits, especially citrus fruits and juices.
vldl cholesterol, calculated
Your reading: 28 mg/dL (normal range 5-40 mg/dL)
VLDL (Very Low Density Lipoprotein) Cholesterol is one of the three major types of lipoproteins that move cholesterol and triglycerides throughout your body. Of these three lipoproteins, VLDL contains the most triglycerides. Like its counterpart LDL, elevated levels of this “bad” cholesterol can increase your risk for heart disease. Healthy lifestyle choices such as regular exercise and good eating habits can lower your triglyceride and VLDL levels.

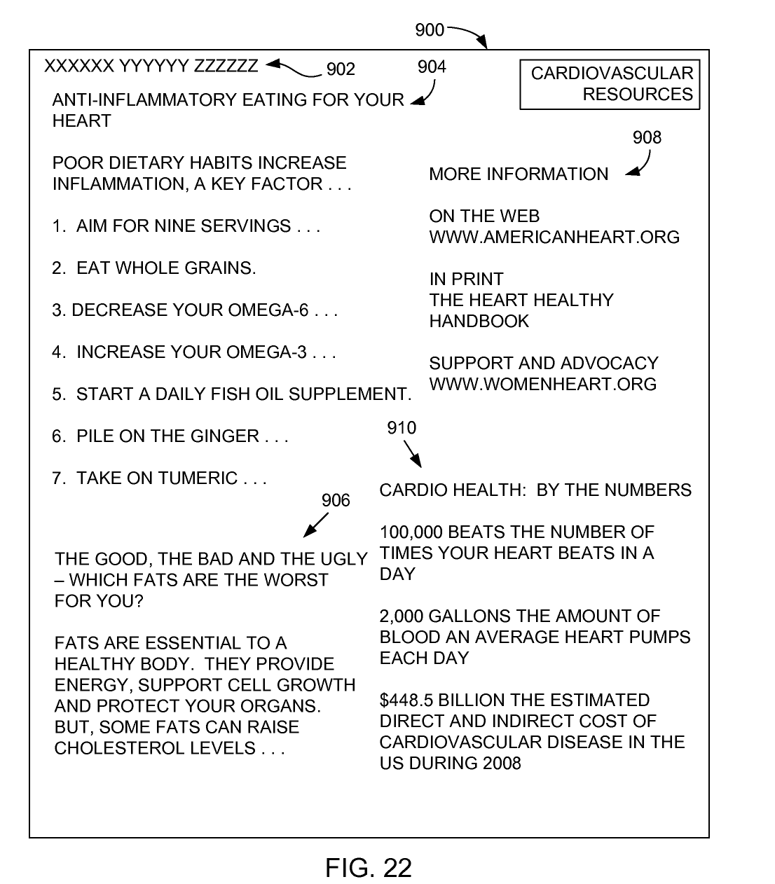

An example lifestyle assessment may include a cardiovascular resources page 900 as illustrated in FIG. 22. An example cardiovascular resources page 900 may include identifying information 902. An example cardiovascular resources page 900 may include one or more sections 904, 906, 908, 910, which may include additional information pertaining to cardiovascular health. For example, section 904 may include the following text:
anti-inflammatory eating for your heart
Poor dietary habits increase inflammation, a key factor in your heart health. Inflammation appears to be central to changes within blood vessels, leading to plaque formation and even rupture. Here are seven ways to incorporate anti-inflammatory agents into your diet.-Dr. Jeffrey Gladd, MD. www.PureHealthMD.com
1. Aim for nine servings of vegetables and fruits per day.
2. Eat whole grains.
3. Decrease your omega-6. Limit products made with soybean, sunflower, safflower and partially hydrogenated oils.
4. Increase your omega-3. Walnuts, flaxseed and fatty fish are good sources.
5. Start a daily fish oil supplement.
6. Pile on the ginger. Try ginger tea, ginger dressing or fresh ginger in stir-fry.
7. Take on tumeric. Try tumeric tea or use this spice when cooking.

Section 906 may include the following text:
The good, the bad and the ugly—which fats are the worst for you?
Fats are essential to a healthy body. They provide energy, support cell growth and protect your organs. But, some fats can raise cholesterol levels and affect your heart health. Do you know which types of fat are the best for you?
The Good: daily caloric intake should be no more than 25-35%
Monounsaturated Fats Contain vitamin E and can help reduce bad cholesterol when eaten in moderation. Found in olive, canola, peanut, sunflower and sesame oils, avocados and peanut butter.
Polyunsaturated Fats Beneficial to your body when eaten in moderation. Found in vegetable and corn oils, walnuts and fatty fish such as salmon.
The Bad: daily caloric intake should be no more than 7%
Saturated Fats Raise cholesterol levels. Found mostly in meat and dairy products, but baked goods and fried foods also contain high levels.
The Ugly: daily caloric intake should be less than 1%
Trans Fats Raise bad cholesterol levels and lowers good cholesterol levels. Found in fried foods, stick margarines, shortenings and some baked goods. Used by restaurants and fast food outlets for frying.
Calculate your daily fat limits at www.americanheart.org

Section 908 may include the following text:
more information
on the web
www.americanheart.org
www.purehealthmd.com
www.hearthealthywomen.org
in print
The Heart Healthy Handbook
A free publication by the National Heart, Lung and Blood Institute
www.nhlbi.nih.gov/health/public/heart
support and advocacy
www.womenheart.org

Section 910 may include the following text:
cardio health: by the numbers
100,000 beats The number of times your heart beats in a day
2,000 gallons The amount of blood an average heart pumps each day
$448.5 billion The estimated direct and indirect cost of cardiovascular disease in the US during 2008
80 million The number of American adults who suffer from cardiovascular disease
50 percent The decrease in smoking among US adults since 1965—smoking is a major risk factor in cardiovascular disease
27 million The number of heart attacks that would be prevented if Americans followed the heart healthy steps outlined by the American Heart Association

Some example lifestyle assessments may include other sections in addition to or instead of the cardiovascular section. For example, some lifestyle assessments may include hypertension, cerebrovascular, metabolism, diabetes, and/or stress sections, each of which may include summary, bioprofile, out of balance reading, in balance reading, and/or resources pages similar to those described above with reference to the cardiovascular section. Some example lifestyle assessments may include a lab report section, in which the readings may be presented in the format of a conventional lab report.

Workplace Report

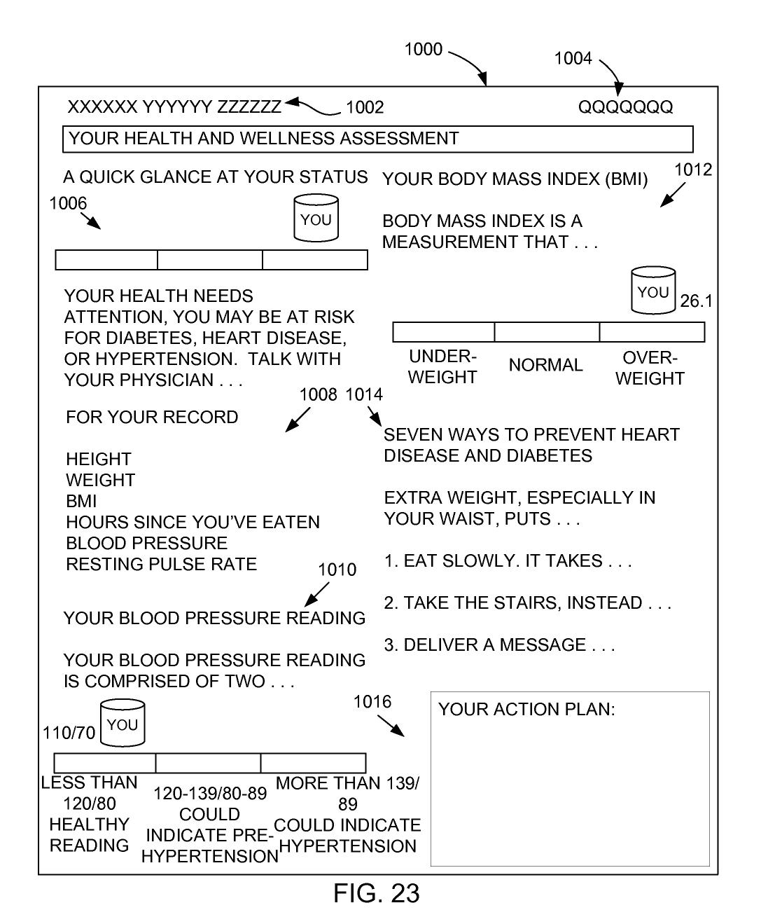

FIG. 23 illustrates an example workplace report 1000, which may include identifying information 1002 (such as, for example, employee name, employee identification number, facility, department, etc.). Some example workplace reports 1000 may include employer information 1004, such as company name, facility, department, etc. Some example workplace reports 1000 may include a summary section 1006, which may provide an overall indication of an employee's health in any manner described herein. For example, some summary sections may include a marker on a scale. Some example summary sections 1006 may include text, such as the following:
Your health needs attention, you may be at risk for diabetes, heart disease or hypertension. Talk with your physician today about your lifestyle habits and ways to improve your test results. It's never too late! Small changes you make today could prevent or even halt future health problems.

Some example workplace reports 1000 may include an other information section 1008, which may include information such as height, weight, body mass index, hours since the patient has eaten (prior having her blood drawn), blood pressure, resting pulse rate, etc.

Some example workplace reports 1000 may include various sections 1010, 1012 providing information pertaining to individual readings. For example, section 1010 may pertain to blood pressure readings and section 1012 may pertain to body mass index. Individual sections 1010, 1012 may include a graphical representation of the reading on a scale in any manner described herein. Example sections 1010, 1012 may include the following text:
your blood pressure reading
Your blood pressure reading is comprised of two numbers. The first is your systolic pressure, which represents the maximum pressure in your arteries when your heart is beating. The second number is your diastolic pressure, which indicates the minimum pressure in your arteries when your heart is resting between beats. Normal blood pressure is critical to good health. High blood pressure contributes to heart disease, stroke and diabetes.
your body mass index (bmi)
Body mass index is a measurement that compares your weight and height. It is helpful for identifying whether you might be under- or over-weight.
your fasting glucose test results
Glucose is an important sugar that is your body's main energy source. After eating, your blood glucose levels increase then gradually decrease over time. Insulin is the hormone that regulates your body's glucose level. Individuals with diabetes are either resistant to or do not produce enough insulin. These test results are most accurate after 8 hours of fasting.
your total cholesterol test results
Cholesterol is a waxy fat-like substance essential to good health, in the right quantities. It has two main sources—75% is manufactured in the body and 25% comes from your food. Two main types of lipoproteins, HDL and LDL, carry cholesterol to and from your liver. LDL is known as the “bad” cholesterol because too much increases your risk of heart disease. HDL is the “good” cholesterol and can protect your heart and arteries.
your hdl cholesterol test results
HDL (High Density Lipoprotein) has been called good cholesterol because it appears to decrease your risk of coronary heart disease. Healthy behaviors like exercise, smoking cessation, and proper diet help increase your HDL and prevent heart disease.
your ldl cholesterol test results
Low-density lipoprotein (LDL) is also known as “bad” cholesterol. High levels of LDL circulating in your blood can slowly build up in the inner walls of the arteries. Over time, this can form blockages in the arteries, possibly resulting in heart attack or stroke. Poor diets, often high in saturated fat can cause elevated LDL levels in the blood.
your triglycerides test results
Triglycerides are the form in which most fat exists in food and the body. They can be found in alcohol, saturated and trans fatty foods and high carbohydrate foods and drinks. A normal amount of triglycerides in your bloodstream is good because it provides energy to your body. However, a high level of triglycerides caused by poor nutrition can increase your risk of heart disease, stroke, obesity and diabetes.

Some example workplace reports may include a health information section 1014, which may include text such as the following:
seven ways to help prevent heart disease and diabetes
Extra weight, especially in your waist, puts you at big risk for diabetes and heart disease. Here are seven small ideas that get you moving and help control your weight.
1. Eat slowly. It takes 20 minutes for your stomach to signal your brain that you're full.
2. Take the stairs, instead of the elevator.
3. Deliver a message to co-worker in person, instead of e-mailing it.
4. Instead of cutting out the foods you love, just cut down on portions and eat them less often.
5. Try to choose foods with little or no added sugar.
6. Pick a parking spot that's furthest from your destination.
7. Don't try to change everything at once! Try one new activity or food a week.
More information: American Diabetes Association,
www.diabetes.org and American Heart Association,
www.americanheart.org
do you know the facts?
You may be making health decisions based on the wrong information. Here are some common myths and the real facts.
Myth: Eating less helps you lose more weight.
Fact: Small and frequent meals keep your metabolism up, burning more calories overall. Too much time between meals causes your body to hoard rather than burn calories.
Myth: There's nothing you can do to prevent Type II Diabetes.
Fact: Studies show that people at risk for Type II Diabetes can prevent or delay onset by losing 5-7% of their body weight.
Myth: If you've smoked a long time, quitting now won't help.
Fact: The minute you quit, you reduce your heart disease risk. Eventually, you can reduce your risk to that of a non-smoker.

Some example workplace reports may include an action plan section 1016, which may allow an employee to write action steps, such as for improving her health.

Although some example embodiments have been described herein as including pages, it is to be understood that some example embodiments may employ one or more pages including the information, sections, graphical representations, etc. as described. Further, as used herein, pages is intended to refer to both hard copy (e.g., paper) documents, as well as electronic representations of the information, sections, graphical representations, etc.

While exemplary embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages may exist even though they may not have been explicitly discussed herein.

What is claimed is:

1. A method for preparing a medical data report, the method comprising:
receiving one or more medical data readings, wherein respective individual medical data readings include a numerical result of a medical test;
processing the medical data readings into report data, wherein the report data includes the individual medical data readings and categorized ranges associated with the medical tests associated with individual medical data readings; and
creating a medical data report including the report data, graphical representations of individual medical data readings and the respective categorized ranges, and textual descriptive information pertaining to the respective medical data readings.

2. The method of claim 1, wherein creating the medical data report includes producing a tangible report for presentation to a patient associated with the medical data readings.

3. The method of claim 1, wherein the categorized ranges associated with individual medical tests include at least two of poor, good, and excellent.

4. The method of claim 3, wherein the graphical representations include distinguishable colors associated with each of the categorized ranges, respectively.

5. The method of claim 1, wherein the graphical representations depict the medical data readings on respective categorized ranges, wherein at least one of the categorized ranges includes a high value and a low value.

6. The method of claim 1, wherein the textual descriptive information includes at least one of a description of a significance of a high reading or a low reading, a suggested action for causing a change in the respective medical data reading, and a suggestion to discuss the respective medical data reading with a medical professional.

7. The method of claim 1, wherein the report includes a health summary page including numbers of readings falling within individual categorized ranges.

8. The method of claim 1, wherein the report includes a detailed health summary page including a bar graph representation of individual medical data readings.

9. The method of claim 8, wherein the detailed health summary page includes instructions pertaining to interpretation of the report.

10. The method of claim 1, wherein the report data, the graphical representations of individual medical data readings and the respective categorized ranges, and the textual descriptive information pertaining to the respective medical data readings are provided on at least one readings page.

11. The method of claim 1, wherein the graphical representations of the individual medical data readings are depicted using a graphical scale.

12. The method of claim 11, wherein the graphical scale includes a bar including medical data indicia along the bar and a representation of at least one of the individual readings also indicated along the bar.

13. The method of claim 11, wherein the graphical scale includes a balance including a first end representing a normal reading and a second end representing a measured reading.

14. The method of claim 13, wherein the balance is tilted towards a greater of the normal reading and the measured reading.

15. A method of communicating medical data to a patient, the method comprising:
processing medical test data into report data, wherein the report data includes individual readings and categorized ranges associated with individual medical tests; and
creating a tangible report that includes, for each medical test, (1) a graphical display of the respective individual reading and the associated categorized ranges and (2) a text description providing information pertaining to the medical test.

16. The method of claim 15, wherein the graphical display includes individual colors associated with the categorized ranges.

17. The method of claim 15, wherein the information pertaining to the medical test includes advice for improving the respective individual reading.

18. The method of claim 17, wherein the advice includes diet advice.

19. The method of claim 15, wherein the report includes a listing of a number of readings associated with individual categories associated with the categorized ranges.

20. The method of claim 19, wherein the report includes a pie chart illustrating relative numbers of readings associated with each of the individual categories.

21. The method of claim 15, wherein the report includes, for individual readings, a bar graph representation of a category associated with the categorized ranges.

22. The method of claim 15, wherein the information pertaining to the medical test includes advice suggesting consultation with a medical professional.

23. The method of claim 15, wherein the report includes a listing of a priority subset of the readings; and wherein the priority subset includes a plurality of readings for which action may be most important.

24. The method of claim 23, wherein the listing of the priority subset of the readings includes an explanation of each of the individual readings comprising the priority subset.

25. The method of claim 15, wherein the individual medical tests include molecular analysis of a biological sample for analytes comprising a molecular bioprofile.

26. The method of claim 25, wherein the molecular bioprofile is produced by identifying a set of relevant bioindicators; correlating the set of relevant bioindicators to create a network; and weighting each of the relevant bioindicators in the set according to its importance.

27. The method of claim 24, wherein molecular analysis of the biological sample include mass spectrometry of a blood sample.

28. The method of claim 15, wherein the molecular analysis of the biological sample includes hematologic analysis of a blood sample.
